(19) United States
(12) Patent Application Publication
(10) Pub. No.: US 2003/0099960 A1
(43) Pub. Date: May 29, 2003

(54) Compositions and methods for optimizing UGT2B7 substrate dosings and for predicting UGT2B7 substrate toxicity

(75) Inventors: Mark J. Ratain, Chicago, IL (US); Federico Innocenti, Chicago, IL (US); Soma Das, Chicago, IL (US); Lalitha Iyer, Santa Clara, CA (US); Michael Sawyer, Edmonton (CA)

Correspondence Address: Gina N. Shishima, FULBRIGHT & JAWORSKI L.L.P., SUITE 2400, 600 CONGRESS AVENUE, AUSTIN, TX, 78701, US

(73) Assignee: The University of Chicago

(21) Appl. No.: 10/057,834

(22) Filed: Jan. 25, 2002

Related U.S. Application Data
(60) Provisional application No. 60/264,534, filed on Jan. 26, 2001.

(51) Int. Cl. C12Q 1/68; A61K 31/70

(52) U.S. Cl. 435/6; 514/23

(57) ABSTRACT

This invention concerns UGT2B7 and its ability to glucuronidate various drugs, including epirubicin. It concerns methods and compositions for determining the level of UGT2B7 activity based on genetic composition, and consequently, allows dosing of UTG2B7-glucuronidated drugs to be improved or optimized based on a patient's level of predicted UGT2B7 activity. It further concerns methods of treatment in which UGT2B7 substrates are administered to patients as part of a treatment regimen.

The present application claims priority to U.S. application Ser. No. 60/264,534, which is specifically incorporated by reference in its entirety.

The government may own rights in the present invention pursuant to grants GM61393 and U01-GM 99-004 from the National Institute of Health.

### BACKGROUND OF THE INVENTION

A. Field of the Invention

The present invention relates generally to the field of cancer therapy. More particularly, it concerns therapeutic and diagnostic methods and compositions concerning optimizing the treatment of cancer patients with epirubicin, and analogs thereof.

B. Description of Related Art

The topoisomerase II inhibitor epirubicin (4′-epi-doxorubicin) is a key component of chemotherapy for breast cancer patients, either in adjuvant or metastatic setting (Omrod et al., 1999). Epirubicin produces similar efficacy with less adverse effects than its analog, doxorubicin, at equimolar doses (Omrod et al., 1999). It is extensively metabolized by the liver, similar to other anthracyclines. Its 13-dihydro derivative, epirubicinol, has very low degree of cytotoxicity, and aglycones of epirubicin and epirubicinol are considered minor inactive metabolites (Schott and Robert, 1989). Epirubicin has a different metabolic fate when compared with doxorubicin, as epirubicin and epirubicinol undergo conjugation with glucuronic acid by liver UDP-glucuronosyltransferase (UGT) enzyme(s) (Weenen et al., 1984).

The main detoxifying pathway for epirubicin is the formation of epirubicin glucuronide (4′-O-β-D-glucuronyl-4′-epi-doxorubicin) (FIG. 1). Among epirubicin metabolites, epirubicin glucuronide is the major metabolite of the drug in plasma as well as in urine (Weenen et al., 1983). Mean area under the plasma concentration-time curve (AUC) values for epirubicin glucuronide were approximately 0.8 to 1.8 times those of the parent drug, while mean AUC values for epirubicinol and its glucuronide were approximately 0.2 to 0.6 times those of epirubicin (Weenen et al., 1983; Mross et al., 1988; Robert and Bui, 1992). Glucuronidation represents a protective mechanism to better eliminate lipophilic xenobiotics and endobiotics from the body, and epirubicin glucuronide is inactive, water soluble and readily excreted in bile and urine (Camaggi et al., 1986).

The UGT isoform that glucuronidates epirubicin had not previously been identified. UGT enzymes are localized in the endoplasmic reticulum and the human isoforms involved in drug metabolism are classified in UGT1 and UGT2 families based on sequence gene homology (Mackenzie et al., 1997). The glucuronidation pathway for epirubicin has been shown to be mainly limited to humans and has been investigated in vitro only in hepatocytes in primary culture (Ballet et al., 1986).

Because epirubicin has a high degree of pharmacokinetic variability among patients (Wade et al., 1992; Robert, 1994), which is unrelated to body surface area (Dobbs et al., 1998), it would be beneficial to be able to modify treatment regimens involving epirubicin or doxorubicin to maximize their efficacy yet minimize their toxicity in individual patients. Identification of polymorphisms in UGT2B7 and screening methods are needed to identify patients at risk for toxicity effects of epirubicin, or analogs of epirubicin, so that dosage and treatment regimens may be altered.

Even more generally, identification of polymorphisms in UGT2B7, including regulatory sequences governing expression, that correlate with glucuronidation activity has significant ramifications regarding any drug that is modified by the polypeptide encoding UGT2B7 in addition to epirubicin, including morphine derivatives, xenobiotics, and many other widely used drugs. While polymorphisms in UGTB7 have been previously identified and investigated, including a polymorphism at amino acid 268 (His or Tyr) (Jin et al., 1993a; Jin et al., 1993b; Mackenzie et al., 2000), no correlation between genotype and phenotype has been observed (Coffman et al., 1998; Bhasker et al., 2000). The observation of such a correlation could be utilized as a screening method to identify toxicity risks and pharmacokinetics of any UGT2B7-glucuronidated drug in particular patients.

### SUMMARY OF THE INVENTION

The present invention relates to determining the level of glucuronidation activity in an individual. Activity may be determined based on the transcript or protein levels of a glucuronidating enzyme, such as UGT2B7. The present invention also concerns genetic screens for directly or indirectly identifying the activity of the the liver glucuronosyltransferase (UGT) enzyme UGT2B7. It concerns determining the general extent to which any UGT2B7-glucuronidated drug will be glucuronidated in a subject that is given or is taking such a drug. It has implications with respect to any drug that is a substrate for UGT2B7 and that can be glucuronidated by UGT2B7 (“UGT2B7-glucuronidated drug” or “UGT2B7 substrate”), including epiribicin. The present invention provides a way of optimizing the dosing for any UGT2B7-glucuronidated drug that has or may be administered to a subject. Accordingly, it also provides a way of addressing toxicity issues related to such drugs. In some embodiments, the present invention addresses the toxicity issue of epirubicin or epirubicin analogs, which are used in the treatment of cancer. It takes advantage of the discovery that UGT2B7 catalyzes the glucuronidation of epirubicin and a number of other well known drugs. The instant invention provides methods and composition for diagnosing persons at risk for epirubicin toxicity or side effects associated with epirubicin, as well as methods and compositions for reducing or eliminating side effects associated with epirubicin treatment, as well as ways of increasing the efficacy of dosage regimens. The methods also apply to other UGT2B7-glucuronidated drugs. It is contemplated that any method or composition (as well as any steps or embodiments) discussed with respect to one UGT2B7-glucuronidated drug, such as epirubucin, may be implemented with respect to any other UGT2B7-glucuronidated drug.

Because epirubicin is administered as a chemotherapeutic, it is contemplated that in many embodiments of the invention, the patient is a cancer patient; however, the present invention applies to any patient who is administered or is taking a UGT2B7-glucuronidated drug. It is contemplated that embodiments disclosed herein with respect to a particular method or composition of the invention may be implemented with respect to other methods or compositions of the invention.

The present invention also takes advantage of the observation that the level of glucuronidation activity of UGT2B7, which modifies a panoply of drugs, is correlated with genotype. Thus, the identification of a patient's genotype provides valuable information regarding the predicted phenotype for that patient with respect to that locus. The invention has broad ramifications for any patient who will be administered or has been administered a drug that is modified by UGT2B7 (UGT2B7 substrate). It has further applications with respect to drug dosage and drug toxicity for UGT2B7 drug substrates.

The present invention, in some embodiments, concerns screening methods that take advantage of pharmacogenetics, which refers to a correlation between a patient's genotype and that patient's phenotype with respect a drug or pharmaceutical compound. In the context of the present invention, pharmacogenetics is relevant to the genotype of UGT enzymes such as UGT2B7 and chemotherapeutic agents, such as epirubicin. It is contemplated that methods described herein with respect to epirubicin may be employed with analogs of epirubicin, all-trans retinoic acid (ATRA)—another anti-cancer drug—and other UGT2B7-glucuronidated drugs.

Thus, in some embodiments of the invention, an assessment can be made about the risk of toxicity from epirubicin in patient depending upon the genotype of the patient's UGT2B7 gene or the phenotype of the patient with respect to UGT2B7 activity and/or expression levels. The term “UGT2B7 gene” refers to the coding (exons) and noncoding regions for UGT2B7. It includes intronic regions, 3′ untranslated regions, and upstream promoter regions, specifically including base−161. In further embodiments a prediction can be made about the degree of epirubicin-induced toxicity in a patient. “Epirubicin-induced toxicity” and “epirubicin toxicity” and “toxicity of epirubicin” are used interchangeably to refer to the toxic effects, as well as symptoms, in pa patient associated with the intake of epirubicin.

In some methods of the invention, there may be a step including identifying a patient at risk for epirubicin-induced toxicity. Methods may also include administering epirubicin to the patient.

In some embodiments, methods involve evaluating the level of UGT2B7 activity or expression in the patient. It is contemplated that a decreased level of UGT2B7 activity or expression is indicative of a patient at risk of epirubicin-induced toxicity. A “decreased level” is relative to an average level found in the general population or to a level found in an average population of patients given epirubicin. UGT2B7 activity refers to the ability of UGT2B7 to glucuronidate a substrate, such as epirubicin. UGT2B7 expression refers to the amount of UGT2B7 protein, though this may be an evaluation based on the amount of UGT2B7 transcripts. Thus, in some embodiments of the invention, the level of UGT2B7 activity is determined in the patient. In others, the level of UGT2B7 expression is determined in the patient. It is contemplated that the level of UGT2B7 expression can be determined by measuring the amount of UGT2B7 transcript or by measuring the amount of UGT2B7 polypeptide. Alternatively, the level of UGT2B activity can be determined by administering a UGT2B7 substrate to a patient and determining the degree of glucuronidation of the substrate. In some embodiments of invention, the substrate is menthol, oxazepam, codeine, naltrexone, naloxone, buprenorphine, ibuprofen, an ibuprofen analog, or morphine.

Because of the pharmacogenetic properties of the UGT enzymes, the present invention also includes determining the level of UGT2B7 activity or expression by evaluating a UGT2B7 gene of the patient for a polymorphism. In some cases, methods of the invention involve evaluating a UGT2B7-coding sequence or a UGT2B7 gene (which includes UGT2B7-coding sequences) for a polymorphism. Such a polymorphism may be in any sequence related to UGT2B7 expression, including a coding sequence, an intron, a control element such as a promoter, or in an untranslated region. In any of the methods described herein involving polymorphisms, more than one polymorphism may be involved. Thus, in some embodiments 1, 2, 3, 4, 5, 6, 7, 8, 9, 10, or more polymorphisms are evaluated and/or identified.

The invention also specifically includes methods for evaluating the epirubicin-induced toxicity in a patient by identifying a polymorphism in a UGT2B7 gene of the patient, wherein the polymorphism results in a decreased level of UGT2B7 activity or expression in the patient. Any of the primers identified as SEQ ID NOS:3-78, inclusive, may be used to identify a polymorphism in a UGT2B7 gene.

The polymorphism in the UGT2B7 gene may be located at position −161, position −125, position +137 position +321, position +372, position +536, position +734, position +801, position +802, position +1059, position +1062, position +1191, position +1288, position +1506, or position +1838 of SEQ ID NO:1, as shown in Table 1. The “A” in the nucleic acid encoding the first methionine (M) of the UGT2B7 polypeptide sequence is designated +1, while nucleotides located upstream of +1 (promoter region) are designated with a “−” to indicate upstream sequence, which is a typical designation for contiguous promoter and coding sequences. For example, the “G” nucleotide adjacent to the 5′ end of the A at +1 is designated “−1.” This “G” also corresponds to position 160 in SEQ ID NO:1. Thus, if a “+” or “−” designation is used with a position number, this indicates the position of a nucleotide relative to the first coding nucleotide (+1). Alternatively, if a position number is designated without the “+” or “−” designation, then the position number is with respect to the 5′ most nucleotide of a given sequence being at position 1.

In some embodiments of the invention, a polymorphism that is evaluated or identified is one that is associated with a decreased level of UGT2B7 activity or expression. Alternatively, a polymorphism may be evaluated for an associated with a decreased level of UGT2B7 activity or expression.

In some embodiments of the invention a polymorphism in a UGT2B7 gene of a patient is identified. In some embodiments the dosage of epirubicin administered to the patient may be adjusted compared to the dosage of epirubicin that would have been administered had a polymorphism in UGT2B7 not been identified in the patient. In other embodiments, a polymorphism results in a decreased level of UGT2B7 activity or expression in the patient. It is contemplated that methods of the invention may also involve comparing the level of UGT2B7 activity or expression in a patient with a UGT2B7 polymorphism to the level of UGT2B7 activity or expression in a patient lacking the polymorphism. In still further embodiments, a polymorphism in a UGT2B7 gene is identified in a sample from a patient, wherein the polymorphism contributes to reduced expression or activity of the UGT2B7 gene product, and a dosage of epirubicin to administer to the patient is determined.

The present invention also includes methods for reducing epirubicin-induced toxicity in a patient. In some embodiments, these are effected by a) evaluating the level of UGT2B7 expression in a sample from a patient; and b) determining a dosage of epirubicin to administer to the patient. In some cases, an evaluation of the level of UGT2B7 expression the patient alters the dosage of epirubicin administered to the patient relative to the dosage that would have been administered to the patient if the level of UGT2B7 expression were higher. Furthermore, the identification of a polymorphism in a UGT2B7 gene may alter the dosage of epirubicin administered to the patient relative to the dosage that would have been administered to the patient if the polymorphism were not identified. In some cases, the dosage of epirubicin administered to the patient may be decreased relative to the dosage that would have been administered to the patient if the polymorphism were not identified, while in other cases the dosage of epirubicin administered to the patient is increased relative to the dosage that would have been administered to the patient if the polymorphism were not identified.

Samples from the patient may be any physical sample that can be evaluated for the patient's genotype or, in some embodiments, for his level of UGT2B7 activity or expression. The sample may be blood, or any other bodily fluid, or a tissue sample or cell culture.

Correlation between genotype and phenotype is one of the touchstones of pharmacogenetics. Identification between a polymorphism and the phenotype it confers is useful information, as it allows for screening of a patient's genotype to yield significant information about the patient's phenotype. The present invention includes methods for identifying a polymorphism in a UGT2B7 gene that identifies a patient at risk for epirubicin-induced toxicity in a patient by: a) obtaining a sample from a cancer patient; b) evaluating a UGT2B7 gene in the sample for a polymorphism; c) administering epirubicin to the patient; and, d) evaluating the patient for epirubicin-induced toxicity. In some embodiments, the patient is administered epirubicin prior to evaluating a UGT2B7 gene in the sample for a polymorphism. Furthermore, the method may include identifying a polymorphism in the UGT2B7 gene.

Identifying a correlation between genotype and phenotype may require a number of data points to be evaluated. With respect to UGT2B7 phenotype, either the level or degree of epirubicin-induced toxicity in a patient may be evaluated or the level of UGT2B7 expression or activity in a patient may be evaluated. Some of the embodiments of the invention involve comparing the UGT2B7 phenotype in a patient against UGT2B7 phenotype in a population of individuals having the polymorphism. The method includes comparing the phenotype observed in the patient against the phenotype seen in a second population of individuals lacking the polymorphism. Alternatively, an average value for either phenotype—level of epirubicin-induced toxicity or level of UGT2B7 activity or expression—may be calculated from patients administered epirubicin, and this may be used as a comparison point against which the significance of an individual's polymorphism(s) may be evaluated. It is contemplated that a general population of patients given epirubicin may be used to provide a baseline against which an evaluation of phenotype, and thus a correlation with a genotype, may be implemented. It is further contemplated that populations of individuals given epirubicin may be subgrouped, particularly when evaluating epirubicin-induced toxicity, depending upon the dosage of epirubicin administered. Thus, dosages for persons within a population may be within 10 mg/m², 20 mg/m², 50 mg/m², or 100 mg/m² of each other.

In other embodiments of the invention, correlation is evaluated in vitro using microsomes carrying a particular UGT2B7 polymorphism. Various polymorphisms may be compared using a glucuronidation assay with epirubicin as a substrate. Level or rate of glucuronidation can be measured to establish a correlation between UGT2B7 genotype and UGT2B7 phenotype.

The present invention is also directed at methods for screening for a modulator of UGT2B7 by: a) incubating a UGT2B7 polypeptide with a substrate under conditions that allow the substrate to be glucuronidated by the UGT2B7 polypeptide; b) incubating the UGT2B7 polypeptide with a candidate substance; and, c) assaying for glucuronidation of the substrate. In some embodiments of the invention, the substrate is epirubicin. It is contemplated that the UGT2B7 polypeptide may be expressed in a host cell comprising a UGT2B7-encoding nucleic acid. In some embodiments, the UGT2B7 polypeptide is isolated away from the host cell prior to incubating the UGT2B7 polypeptide with the substrate. Also, the UGT2B7 polypeptide may be comprised in a liver microsome expressing UGT2B7.

Other methods of identifying a UGT2B7 modulator include: a) determining a standard transcription and/or translation activity profile of a UGT2B7 nucleic acid sequence; b) contacting the UGT2B7-encoding nucleic acid segment with a candidate substance; c) maintaining the nucleic acid segment and candidate substance under conditions that allow for UGT2B7 transcription and translation; and d) assaying for a change in the transcription and/or translation activity. A standard transcription or translation profile refers to an average amount of transcription or translation observed under similar conditions but without the candidate substance.

Modulators of UGT2B7 may be UGT2B7 inducers, such as ones that increase UGT2B7 transcription, increase the amount of UGT2B7, or increase its activity. Alternatively, the modulator may be UGT2B7 or a UGT2B7-encoding nucleic acid themselves since providing either may result in an increase in the amount of UGT2B7 or an increase in UGT2B7 activity in a cell or in a cell free system.

Methods are contemplated using UGT2B7 modulators. They includes methods for reducing epirubicin-induced toxicity or the risk of epirubicin-induced toxicity comprising administering epirubicin to a patient in combination with a UGT2B7 modulator that increases UGT2B7 activity in the patient. A UGT2B7 modulator may be identified by any methods described herein.

In some embodiments, epirubicin, or another compound such as a modulator or second agent, is administered parenterally, including by intravenous injection or by bolus intravenous injection; in others, they may be administered orally, or by any other route described herein.

In further embodiments of the invention, there are methods of treating a patient with cancer, comprising administering to the patient a therapeutically effective combination of a epirubicin drug and a second agent that reduces excretion of the active epirubicin species through the bile. In still further embodiments, methods include administering to the patient a therapeutically effective combination of epirubicin drug, a second agent that increases conjugative enzyme activity and a third agent that decreases biliary transport protein activity. “Therapeutically effective” refers to an ability to effect a therapeutic result. “Effective amount” refers to an amount that can effect a particular result, such as increase glucuronidation of epirubicin. With the methods of the present invention, a second agent may be administered to the patient prior to the epirubicin drug. In some embodiments, a second agent increases the activity of a conjugative enzyme or decreases the activity of a biliary transport protein, while in other embodiments, a second agent increases glucuronosyltransferase enzyme activity. A second agent can comprise a nonsteroidal anti-inflammatory agent or t-buthylhydroquinone. Nonsteroidal anti-inflammatory agent include indomethacin, sulindac, tolmetin, acemetacin, zopemirac, and mefenamic acid.

Compositions of the invention include those comprising an epirubicin drug in combination with a UGT2B7 modulator, which can be dispersed in a pharmacologically acceptable formulation.

Moreover, the present invention encompasses kits comprising a pharmaceutical formulation of a epirubicin drug and a pharmaceutical formulation of a UGT2B7 modulator that increases UGT2B7 activity or expression level, in suitable container means. In some embodiments, epirubicin and the modulator are present within a single container means, though they may be present within distinct container means. It is contemplated that pharmaceutical formulations are suitable for parenteral or oral administration. Other kits of the invention include kits that allow for identification of UGT2B7 polymorphisms. They may include any of the primers described herein, and in some embodiments include other reagents that allow for screening of polymorphisms.

Aspects of the invention are directed to any drug that can be glucuronidated by UGT2B7 (any variant or polymorphism) (referred to as “UGT2B7 substrate” or “UGT2B7 glucuronidated substrate”). Such aspects concern methods and kits. It is contemplated that any embodiment described herein with respect to epirubicin may be implemented with respect to any UGT2B7 substrate and vice versa, and that a person of ordinary skill in the art would be able to practice such embodiments.

The present invention also concerns methods for predicting the level of glucuronidation in a patient. In some cases, it involves determining or predicting the level of glucuronidation of a UGT2B7 substrate in a patient comprising determining the nucleotide sequence of base −161 in one UGT2B7 promoter of the patient. This will allow the dosing for a particular UCT2B7-glucuronidated drug to be determined. Methods involve a) determining the nucleotide sequence at position −161 in one UGT2B7 gene of the patient, which may be done directly (identifying the sequence of position −161) or indirectly (identifying the sequence of one or both alleles of a polymorphism in complete linkage desequilibrium with polymorphism −161). In further embodiments, methods include b) classifying the UGT2B7 activity level in the patient, whereby identification of a thymidine residue indicates the patient does not have a low level of activity and/or determining the dose of the UGT2B7-glucuronidated drug to prescribe to the patient based on the sequence at position −161 of the UGT2B7 gene. In further embodiments, determining the level of UGT2B7 activity or expression (transcript or polypeptide) involves determining the nucleotide sequence at position −161, +801, and/or +802 in the UGT2B7 gene.

In some embodiments of the invention, methods concern a patient who has or will be administered a UGTB7-glucuronidated drug. Such patients may have been or will be given such a drug within 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, or 1, 2, 3, 4, 5, 6, 7, 8, 9, 10, 11, 12 or more weeks. It is further contemplated that the patient will not be given a particular UGT2B7-glucuronidated drug because of the level of UGT2B7 activity determined in that patient.

In some embodiments, the nucleotide sequence of base −161 in both UGT2B7 promoters of the patient are determined. The level of glucuronidation activity of UGT2B7 with respect to a UGT2B7 substrate can be predicted depending upon the sequence of base −161 in the promoter for the gene encoding UGT2B7. As discussed herein, patients with thymidine residues at position −161 in both UGT2B7 promoters will be considered to have the highest level of UGT2B7 activity (“high glucuronidators”); patients with one thymidine residue and one cytosine residue at position −161 in each UGT2B7 promoter have the next highest level of UGT2B7 activity (“intermediate glucuronidators”); and, patients with cytosine residues at position −161 in both UGT2B7 promoters have the lowest level of UGT2B7 activity (“low glucuronidators”). Therefore, persons with a T/T genotype at position −161 are considered to have a high level of UGT2B7 activity, persons with a C/T genotype at that position are considered to have an intermediate level of UGT2B7 activity, and persons with a C/C genotype at position −161 are considered to have a low level activity (when a base from only one promoter is known, it will be known that the person is an intermediate or high glucuronidator if that one nucleotide is a T, while a person with one identified base at −161 that is a C is an intermediate or low glucuronidator). This idea is generally understood to mean that the average for persons with a high level of activity is higher than the average for persons with an intermediate level of activity and that the average for persons with an intermediate level of activity is higher than the average of persons with a low level of activity. It is further contemplated that such qualifications may be assessed based on a random sampling of the general population (that is, more than 100 persons).

“UGT2B7 activity” in the context of a patient refers to the overall glucuronidation activity of the polypeptide encoded by the UGT2B7 gene in a patient (as opposed to its activity with respect to individual substrates). A patient's level of UGTB7 activity can be assessed by evaluating the genotype of the UGT2B7 gene or by evaluating the amount of UGTB7 transcript or polypeptide levels. Experimental evidence shows that the activity of any UGTB7 polypeptide as opposed to the overall activity of UGTB7 in a patient is relatively constant. However, it should be noted that UGT2B7 has different binding specificities to its various substrates (reflected in Kₘ), and thus, its activity may be generally qualified (for example, in terms of Vₘₐₓ, or specifically determined with respect to a particular substrate (referred to as “UGT2B7 specific activity”).

In some embodiments of the invention, methods include obtaining a sample from the patient, using the sample to determine the nucleotide sequence of the nucleotide at position −161 of the UGT2B7 promoter.

The invention includes embodiments in which determining the nucleotide sequence of base −161 in the UGT2B7 promoter involves amplifying a sequence from the UGT2B7 promoter or from the UGT2B7 coding region (amplifying a polymorphism in coding region that is in complete linkage disequilibrium with −161 polymorphism). In other embodiments, the invention includes determining the nucleotide sequence of base −161 in the UGT2B7 promoter by sequencing a portion of the UGT2B7 promoter, for example, a portion comprising base −161 or sequencing a portion of the UGT2B7 gene (promoter, introns, or exons) that covers a polymorphism in complete linkage disequilibrium with the polymorphism at −161, such as the first nucleotide of codon 268 (nucleotide +802). Complete linkage disequilibrium (LD) means, for example, that when the nucleic acid sequence at −161 is a “T” (nucleotide), the sequence at +802 is a “T” in 100% of the samples evaluated. Similarly, when a “C” was observed in one strand at −161, a “C” was observed in one strand 100% of the time at +802. Determining the nucleotide sequence of base −161 can also be done by determining the nucleotide sequence of other sequences in complete LD with −161 or any of the polymorphisms that are in complete LD with −161. Such polymorphisms include +801 (third nucleotide of codon 267), which is in complete LD with nucleotide +802. A “T” nucleotide at +801 is in complete linkage disequilibrium with a “C” nucleotide at +802, while an “A” nucleotide at +801 is in complete linkage disequilibrium with a “T” at +802, which has been previously described. Consequently, −161 and +801 are in complete LD with each other. A “C” at −161 indicates a “T” at +801, while a “T” at −161 means an “A” at +801. Thus, in some embodiments of the invention, determining the nucleotide sequence of base −161 in the UGT2B7 promoter can be done by determining the sequence of a polymorphism that is in complete linkage disequilibrium with it. In further embodiments of the invention, methods of predicting the level of glucuronidation activity or the amount of UGT2B7 (transcript, protein, or activity) can be accomplished by determining the genetic sequence of the these polymorphisms in complete LD with polymorphism −161, using the same methods as with −161. Furthermore, embodiments of the invention comprise methods in which the sequence of more than one polymorphism (either more than one strand of a single polymorphism or different polymorphisms) is identified. Thus, the present invention includes methods in which one or both strands of 1, 2, 3, 4, or more polymorphisms in complete LD with −161 (including −161) are identified.

As discussed above, methods include also determining the nucleotide sequence at position −161 in a second UGT2B7 gene in the patient, whereby 1) identification of a second thymidine residue indicates the patient will have a high level of UGT2B7 glucuronidation (capabilities); 2) identification of a second cytosine residue indicates the patient will have a low level of UGTB7 glucuronidation; and/or, 3) identification of a residue different than the residue in the first promoter (C/T or T/C) indicates an intermediate level of glucuronidation. It is contemplated that identification of at least one “C” residue indicates the person has either low or intermediate levels of UGT2B7 glucuronidation capabilities. In still further embodiments of the invention, methods of determining level of glucuronidation comprise the step of classifying the UGT2B7 activity level of the patient based on the sequence of one or more nucleotides in the UGT2B7-encoding and -regulating sequence. A UGT2B7-regulating sequence refers to those nucleotides that contribute or affect the level of UGT2B7 transcript, protein, or activity in a cell, including, but not limited to promoter, enhancer, and intronic sequences for UGT2B7.

In some embodiments of the invention, patients may be classified according to their predicted level of UGT2B7 activity (or transcript or protein level). In othe embodiments of the invention, a patient may first be identified in need of a UGT2B7-glucuronidated drug, and then the method of determining the level of UGT2B7 activity be implemented. Alternatively, a person may be identified as needing to have his or her level of UGT2B7 glucuronidation determined either prior to or after administration of a UGT2B7-glucuronidated drug. The determination may be part of a physician's decision whether to administer a particular UGT2B7-glucuronidated drug to the patient or in his/her decision as to which such drug to give the patient. It may also be part of the physician's determination not whether to administer a UGT2B7-glucuronidated drug, but at what dose or dosage (amount and/or frequency) to administer it. Finally, it may be part of a physician's decision about whether to administer other drugs in conjunction with the regimen to administer a UGT2B7-glucuronidated drug, for example, to reduce the side effects or toxicity of the UGT2B7-glucuronidated drug.

Further embodiments of the invention concern determining the nucleotide sequence of a first polymorphism in complete linkage disequilibrium (LD) with base −161 of the UGT2B7 promoter as a way of determining the sequence of base −161. In some cases, sequencing involves determining the nucleotide sequence of the first base in the codon encoding residue 268 in a UGT2B7 polypeptide. If the nucleotide at +802 is a cytosine in one strand, then the base at −161 will be a cytosine in one strand; if a nucleotide at +802 is a thymidine in one strand, then the base at position −161 will be a thymidine in one strand, and vice versa. Complete LD may also be the case for these positions and position +801 (C to A). If there is a C in one strand at either position −161 or +802, there will be a C at +801; if there is a T in one strand at either position −161 or +802, there will be an A at +801. Other polymorphisms identified herein may also be in complete LD with −161 and +802. The first base of the codon encoding residue 268 is a cytosine in some embodiments, while in others, it is a thymidine. Additional embodiments involve determining the nucleotide sequence of base −161 in one UGT2B7 promoter by determining the nucleotide sequence of a second polymorphism or another polymorphism in complete linkage disequilibrium (LD) with base −161 of the UGT2B7 promoter. This polymorphism could be the other allele of the first polymorphism in complete LD with base −161 or it could be a different polymorphism in complete LD with −161. Such polymorphisms include +801 (third nucleotide of codon 267), which is in complete LD with nucleotide +802. A “T” nucleotide at +801 is in complete linkage disequilibrium with a “C” nucleotide at +802, while an “A” nucleotide at +801 is in complete linkage disequilibrium with a “T” at +802, which has been previously described. Consequently, −161 and +801 are in complete LD with each other. A “C” at −161 indicates a “T” at +801, while a “T” at −161 means an “A” at +801.

UGT2B7 chemically modifies (glucuronidates) a number of substrates. These include compounds with aliphatic carboxylic acids functions, such as NSAIDs and other pain relievers, hormones, xenobiotics, opioids and opioid derivatives, and endogenous compounds. Substrates are administered to patients as drugs in embodiments of the invention. Any of these could be administered to a patient and the UGT2B7 activity in that patient would be relevant to toxicity, effective dosage, clearance, and/or side effects generally. Thus, the present invention has applications with respect to any UGT2B7 substrate, including, but not limited to, those identified herein. Furthermore, any of these substrates can be used to determine phenotypic correlation between UGT2B7 genotype and phenotype or activity of UGT2B7 polypeptide with respect to that substrate.

Compounds with an aliphatic carboxylic acid function include a propionic acid derivative, a phenylacetic acid derivative, a salicylic acid derivative, a acetic acid derivative, or an isobutyric acid derivative. A proprionic acid derivative includes benoxaprofen, fenoprofen, ketoprofen, ibuprofen, naproxen, or tiaprofenic acid. A phenylacetic acid derivative includes etodolac, oxaprozin, or zomepirac. A salicylic acid derivative includes diflunisil. An acetic acid derivative includes indomethacin, valproic acid, or zomepirac. An isobutyric acid derivative includes clofibric acid. Other substrates are polyhydroxylated estrogens, including 4-hydroxyestrone, estriol, or 2-hydroxyestriol. Xenobiotic substrates include 2-aminophenol, 4-OH biphenyl, androsterone, 1-naphthol, 4-methylumbelliferone, menthol, 4-nitrophenol, or hyodeoxycholic acid. Opioid substrates could be morphinan derivatives, including normorphine, norcodeine, codeine, naloxone, nalorphine, naltrexone, oxymorphone hydromorphone, dihydromorphone, levorphanol, nalmefene, naltrindole, naltriben, nalbuphine, morphine (3-glu or 6-glu). Other opioid substrates are oripavine derivatives, including norbuprenorphine, buprenorphine, or diprenorphine. Additional UGT2B7 substrates are propranolol, temazepam, chloramphenicol, oxazepam, androsterone, epitestosterone, epitestosterone, zidovudine (AZT), or all-trans retinoic acid (ATRA), as well as those identified in Radominska-Pandya et al., 2001, which is hereby incorporated by reference. Cyclosporine A and tacrolimus are also UGT2B7 substrates and may be used in any embodiment of the invention (Strassburg et al., 2001). As discussed above, epirubicin is a substrate for UGT2B7. The hydroxyl metabolites of anthracyclines also may be substrates for UGT2B7 and thus methods and compositions of the invention apply to them as well.

Other methods of the invention concern methods of treating a patient with or methods of determining drug dosages or doses of UGT2B7 substrates that are used as drugs in patients. These embodiments involve predicting the activity level of UGT2B7 in a patient and determining a dose of the drug to administer to the patient based on whether the patient has a high, medium, or low level of UGT2B7 activity. It is specifically contemplated that methods described with respect to predicting UGT2B7 activity levels may be implemented in conjugation with methods of treating patients or methods of determining drug dosage for a patient. In further embodiments of the invention, a dosage or drug that may have been given to a patient without knowing his or her UGT2B7 activity level is modified based on the patient's predicted UGT2B7 activity level. The dosage may be increased or decreased, or not given at all, or the patient may be given a different drug because of his or her UTG2B7 activity level.

In additional embodiments of the invention, methods for evaluating the risk of toxicity of a UGT2B7-glucuronidated drug in a patient are contemplated. They comprise: a) identifying a patient in need of evaluation of the risk of toxicity of a UGT2B7-glucuronidated drug; b) obtaining a sample from the patient; c) determining the nucleotide sequence at position −161 in one UGT2B7 gene of the patient. The sample may be from any source (blood, tissue, serum, other bodily fluid) so long as it contains genomic DNA and/or RNA transcripts.

In still further methods of the invention, methods of screening an individual for glucuronidation activity is included. Such methods comprise a) identifying a patient in need of screening for glucuronidation activity; and, b) identifying the nucleotide sequence of a polymorphism that correlates with glucuronidation activity in the individual. As described herein, polymorphisms described herein, including those at positions −161, +801, or +802 in the UGT2B7 gene qualify. As described throughout the specification, polymorphism can be identified by amplifying the nucleic acid by PCR or by sequencing the nucleic acid in the relevant region.

Other methods involve prescribing a dose of a UGT2B7-glucuronidated drug to a patient comprising: a) obtaining a sample from a patient in need of the UGT2B7-glucuronidated drug; and b) determining the level of UGT2B7 glucuronidation in the patient.

Another embodiment of the invention is a kit, in a suitable container means, that can be used to predict UCT2B7 activity in a patient. In some embodiments, the kit includes reagents for determining the nucleic acid sequence at position −161 of one or two UGT2B7 promoters. Thus, primers for amplification reactions or other nucleic acid detection reagents are included. In some embodiments, kits for evaluating the level of UGT2B7 activity in a subject may include, in a suitable container means, a first, second, and/or third nucleic acid comprising 15 contiguous bases complementary or identical to the UGT2B7 gene, wherein the nucleic acid allows the identification of the sequence of a polymorphism in the UGT2B7 gene. The nucleic acids may allow identification of different polymorphisms (i.e., different positions, not different alleles) at −161, +801, and +802. In further embodiments, the nucleic acids are attached to a nonreactive array plate. Identification of the allele(s) of a polymorphism may be accomplished by methods well known to those of skill in the art, for example, by using nucleic acid amplification, detection reagents (colorimetric, radioactive, enzymatic, or fluorimetric), and nucleic acid sizing methods (electrophoresis).

As used herein, “any integer derivable therein” means a integer between the numbers described in the specification, and “any range derivable therein” means any range selected from such numbers or integers.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Structural formula and metabolic pathways of epirubicin in humans. The ketone moiety of C-13 is reduced in epirubicinol, and the hydroxyl group of C-4′ is axial in doxorubicin and equatorial in epirubicin, which allows conjugation of epirubicin with glucuronic acid. The transformation of epirubicin in its glucuronide (big arrow) represents the major detoxifying pathway.

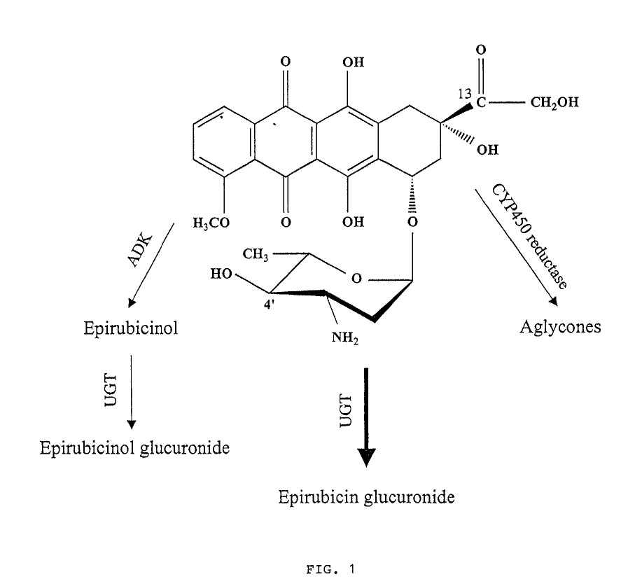

FIGS. 2A-2B. Michaelis-Menten kinetics of glucuronidation of epirubicin by normal liver microsomes (A) and UGT2B7 microsomes (B). Pooled human liver microsomes and UGT2B7 microsomes (3 mg/ml) were incubated for 4 h in the presence of 5 mM UDPGA and increasing amount of epirubicin (range, 50-1000 μM). Data are shown as mean±SD of two separate experiments performed in triplicate.

FIG. 3. Frequency distribution of epirubicin glucuronidation in 47 microsomes preparations from normal human liver donors. This phenotype is normally distributed.

FIGS. 4A-C. Correlation analysis between formation rates of epirubicin glucuronide versus those of M3G (A), M6G (B), and SN-38 glucuronide (C) in 47 normal human liver microsomes. Epirubicin glucuronidation is significantly related to that of M3G (r=0.76, p<0.001) and M6G (r=0.73, p<0.001). No evidence of correlation is observed between epirubicin and SN-38, a substrate of UGT1A1 (r=0.04).

FIG. 5. Frequency distribution of ratios of morphine 6 glucuronide to morphine.

### DESCRIPTION OF ILLUSTRATIVE EMBODIMENTS

The present invention relates methods and composition for reducing the toxicity of the anti-cancer drug, epirubicin and its analogs, as well as methods and compositions for optimizing the dosage/treatment regimens of epirubicin and its analogs in patients. The inventors have determined that epirubicin is glucuronidated by the UGT isoform, UGT2B7. Embodiments of the present invention therefore relate to methods and compositions for identifying patients at risk for toxicity effects of epirubicin, and analogs thereof, as well as for reducing those effects.

I. Epirubicin

Epirubicin, also marketed as Pharmorubicin® or Ellence™, is an antineoplastic drug of the anthracycline class and is a 4′-epimer of doxorubicin. Epirubicin works by the inhibition of topoisomerase II, thereby affecting cellular DNA, which leads to its cytotoxicity.

Epirubicin is indicated as a component of adjuvant therapy for patients with various types of cancers including breast cancer, lung cancer, ovarian carcinoma, soft-tissue sarcomas, other solid neoplasms and hematological malignancies. The overall efficacy of the drug is comparable to doxorubicin, although an important feature is reduced cardiotoxicity in comparison to doxorubicin. Increased cardiac tolerability allows the administration of both, larger dosages of epirubicin per therapy as well as increases the number of administrations of the drug. Hence, epirubicin based treatments provide an alternative to doxorubicin when anthracycline based therapies are sought.

The metabolism of epirubicin results in the formation of relatively inactive to totally inactive metabolites including a 13-dihydro derivative, epirubicinol, two glucuronides and four aglycones. The glucuronides of epirubicin and epirubicinol are quantitatively important and the pathway of glucuronidation mediated by specific enzymes is responsible for better tolerability of the drug.

Elimination of the epirubicin is primarily biliary, with less than 15% being excreted in the urine. Drug pharmacokinetics are described by a 3-compartment model with median half-life values of about 3.2 minutes, 1.2 hours and 32 hours for each phase. The total plasma clearance is about 46 L/h/m². Maximum tolerated doses are about 150 to 180 mg/m².

A. Route and Dosage

Epirubicin is generally administered intravenously (i.v.), although other routes of administration are also possible. In adults, about 100 to 120 mg/m2 intravenous (I.V.) infusion over 3 to 5 minutes via a free-flowing I.V. solution on day 1 of each cycle every 3 to 4 weeks, or divided equally in two doses on days 1 and 8 of each cycle. The cycle can be repeated every 3 to 4 weeks for six cycles and used concurrently with regimens containing cyclophosphamide and 5-fluorouracil.

Dosage modification after the first cycle is generally based on toxicity. For patients experiencing platelet counts <50,000/mm³, absolute neutrophil count (ANC)<250/mm³, neutropenic fever, or grade 3 or 4 nonhematologic toxicity, the day 1 dose in subsequent cycles are reduced to about 75% of the day 1 dose given in the first cycle. Day 1 therapy in subsequent cycles is generally delayed until platelets are >100,000/mm³, ANC>1,500/mm³, and nonhematologic toxicities recover to grade 1.

For patients receiving divided doses (days 1 and 8), the day 8 dose is about 75% of the day 1 dose if platelet counts are 75,000 to 100,000/mm³ and ANC is 1,000 to 1,499/mm³. If day 8 platelet counts are <75,000/mm3, ANC <1,000/mm³ or grade 3 or 4 non-hematologic toxicity occurs, day 8 doses are omitted.

Dosage adjustments are performed in patients with bone marrow dysfunction (For example, heavily pretreated patients, patients with bone marrow depression, or those with neoplastic bone marrow infiltration). Such patients are typically started at lower doses of 75 to 90 mg/m2. For patients manifesting hepatic dysfunction, if bilirubin is 1.2 to 3 mg/dl or aspartate aminotransferase (AST) is two to four times upper limit of normal, one-half of the recommended starting dose is administered. If bilirubin is >3 mg/dl or AST is >four times upper limit of normal, one-quarter of the recommended starting dose is administered. In patients with severe renal dysfunction with serum creatinine >5 mg/dl, lower dosages are considered.

B. Adverse Reactions

Some of the adverse effects (side effects) seen with epirubicin are lethargy, cardiomyopathy, heart failure, conjunctivitis, keratitis, nausea, vomiting, diarrhea, anorexia, mucositis, amenorrhea, leukopenia, neutropenia, febrile neutropenia, anemia, thrombocytopenia, alopecia, rash, itch, skin changes, fever, hot flashes, and other forms of local toxicity.

C. Metabolism of Epirubicin

Epirubicin is predominantly metabolized by the liver, however, other organs and cells such as the red blood cells also participate in its metabolism. A variety of enzymes participate in the metabolism of epirubicin including aldoketoreductases, which produce a 13-dihydro metabolite; and glucuronosyltransferases. The glucuronosyltransferases appear to be unique to the human metabolism of epirubicin, as these enzymes and their metabolites have not been seen in studies on animal models.

This unique metabolic pathway, first described by Weenen et al., 1983, and 1984, produces glucuronic acid conjugates of epirubicin and epirubicinol in the plasma and urine of patients treated with epirubicin. These types of metabolites are non-toxic and are unique to epirubicin. For example, in the closely related drug, doxorubicin, such conversion is not possible due to the lack of the 4′ equatorial orientation of a hydroxyl moiety at the C4 position. This type of metabolism accounts largely for the lower toxicity of epirubicin in comparison to doxorubicin. Other antineoplastic agents that are eliminated by glucuronidation include but are not limited to camptothecins like SN-38.

D. Anthracyclines

Epirubicin is an anthracycline. Except for alkylating agents, anthracyclines have the most significant breadth with respect to their antitumor spectrum. Anthracyclines are used as anticancer agents against various types of cancers including breast cancers, sarcomas, Hodgkin's and non-Hodgkin's lymphomas, pediatric solid tumors, myelomas, acute lymphocytic and myeloid leukemias, stomach carcinomas, small cell carcinomas, ovarian cancers, endometrial carcinomas, transitional cell carcinomas, thyroid carcinomas, non-small-cell carcinomas of the lung, and carcinoid and malignant thymomas. In addition, the anthracycline, doxorubicin in its lyposome encapsulated form has antineoplastic effects in AIDS-related Kaposi's sarcoma.

It is contemplated that other anthracyclines and related drugs, such as anthracenediones may be substrates for UGT family members, particularly UGT2B7. Anthracyclines include doxorubicin, daunorubicin, 4-demethoxydaunorubicin, MEN 10755, MEN 11463, MEN 11951, MEN 10959, idarubicin, pirarubicin, mitoxantrone, annamycin, daunosamine, acosamine, ristosamine, epi-daunosamine, carmynomicin, and KRN8602. However, it is already known that doxorubicin is not glucuronidated. These other anthracyclines may be evaluated as substrates for UGT2B7 in screening assays of the present invention.

II. Glucuronosyltransferases and UGT2B

Glucuronidation is the process by which glucuronic acid is attached to toxic compounds to facilitate their elimination. Glucuronosyltransferases such as the UDP-glucuronosyltransferases (UGT) catalyze this process. UGTs are intrinsic membrane proteins of the endoplasmic reticulum and the nuclear envelope and are encoded by genes of at least two gene families, the UGT1 and UGT2 gene families. The UGT1 gene family members are encoded by a complex gene composed of several exons. UGT1 gene products often share common second to fifth exons and have at least another twelve exons that give rise to a large repertoire of proteins with unique N-terminal domains by alternative splicing. The UGT2 gene products are transcribed from unique genes. Several isoforms of UGT have been identified with the UGT2B7 isoform being very important in humans.

The UGT2B7 isoform catalyzes the glucuronidation of several drugs such as the opioid analgesics, for example, morphine, codeine, and buprenorphine with high efficiency (Coffman et al., 1997). Coffman et al. (1997), have also shown that UGT2B7 also catalyzes the glucuronidation of certain androgenic steroids, various xenobiotics, menthol, propranolol, oxazepam and the like. UGT2B7 chemically modifies a number of substrates, including, but not limited to, compounds with aliphatic carboxylic acids functions, such as NSAIDs and other pain relievers, hormones, xenobiotics, opioids and opioid derivatives, and endogenous compounds. Compounds with an aliphatic carboxylic acid function include a propionic acid derivative, a phenylacetic acid derivative, a salicylic acid derivative, a acetic acid derivative, or an isobutyric acid derivative. A proprionic acid derivative includes benoxaprofen, fenoprofen, ketoprofen, ibuprofen, naproxen, or tiaprofenic acid. A phenylacetic acid derivative includes etodolac, oxaprozin, or zomepirac. A salicylic acid derivative includes diflunisil. An acetic acid derivative includes indomethacin, valproic acid, or zomepirac. An isobutyric acid derivative includes clofibric acid. Other substrates are polyhydroxylated estrogens, including 4-hydroxyestrone, estriol, or 2-hydroxyestriol. Xenobiotic substrates include 2-aminophenol, 4-OH biphenyl, androsterone, 1-naphthol, 4-methylumbelliferone, menthol, 4-nitrophenol, or hyodeoxycholic acid. Opioid substrates could be morphinan derivatives, including normorphine, norcodeine, morphine, codeine, naloxone nalorphine, naltrexone, oxymorphone hydromorphone, dihydromorphone, levorphanol, nalmefene, naltrindole, naltriben, nalbuphine, morphine (3-glu), morphine (6-glu), or UDP-GlcUA. Other opioid substrates are oripavine derivatives, including norbuprenorphine, buprenorphine, or diprenorphine. Additional UGT2B7 substrates are propranolol, temazepam, chloramphenicol, oxazepam, androsterone, or epitestosterone, as well as those identified in Radominska-Pandya et al., 2001, which is hereby incorporated by reference. Cyclosporine A and tacrolimus are also UGT2B7 substrates and may be used in any embodiment of the invention (Strassburg et al., 2001). The hydroxyl metabolites of anthracyclines also may be substrates for UGT2B7 and thus methods and compositions of the invention apply to them as well.

The present inventors have demonstrated herein that epirubicin (EPI) is converted into epirubicin glucuronide (EPI-G) by the UGT2B7 isoform. Thus, the discovery that UGT2B7 is responsible for the conversion of epirubicin into a less toxic version provides a variety of compositions and methods described herein for use in the evaluating and reducing the risk of toxicity of epirubicin, and analogs thereof, in patients given epirubicin and epirubicin analogs as a treatment regimen. Methods and compositions involving screening for modulators of UGT2B7 activity and expression, as well as the modulators themselves, also take advantage of the inventors' discovery. These various methods and compositions involving UGT2B7, such as UGT2B7 nucleic acid molecules, UGT2B7 proteinaceous compositions, which are discussed in further detail below.

Polymorphisms and single nucleotide polymorphisms (SNPs) have been identified in the UGT2B7 gene. Some of these are taught in WO 0006776, which is specifically incorporated by reference. The discovery of some polymorphisms is also described herein. A list of polymorphisms is provided in Table 1.

TABLE 1

| Polymorphism | Location |
|---|---|
| −161 T/C | Promoter:161 bp upstream of the ATG start |
| | site |
| −125 T/C | Promoter:125 bp upstream of the ATG start |
| | site) |
| +137 T/C | Exon 1 |
| +321 T/A | Exon 1 |
| +372 G/A | Exon 1 |
| +536 C/T | Exon 1 |
| +735 G/A | Exon 2 |
| +801-802 TC/AT | Exon 2 |
| +1059 G/C | Exon 4 |
| +1062 T/C | Exon 4 |
| 154 ΔA | Intron 4 |
| +1191 C/T | Exon 5 |
| +1288 A/C | Exon 5 |
| +1506 A/G | Exon 6 |
| +1838 C/A | Exon 6 |

Numbering designations are described in Summary of Invention.

A. Nucleic Acids

The present invention involves nucleic acids, including UGT2B7-encoding nucleic acids, nucleic acids identical or complementary to all or part of the sequence of a UGT2B7 gene, nucleic acids encoding modulators of UGT2B7 and the UGT2B7 gene, as well as nucleic acids constructs and primers.

The present invention concerns polynucleotides or nucleic acid molecules relating to the UGT2B7 gene and its gene product UGT2B7. These polynucleotides or nucleic acid molecules are isolatable and purifiable from mammalian cells. It is contemplated that an isolated and purified UGT2B7 nucleic acid molecule, that is a nucleic acid molecule related to the UGT2B7 gene product, may take the form of RNA or DNA. As used herein, the term “RNA transcript” refers to an RNA molecule that is the product of transcription from a DNA nucleic acid molecule. Such a transcript may encode for one or more polypeptides.

As used in this application, the term “polynucleotide” refers to a nucleic acid molecule, RNA or DNA, that has been isolated free of total genomic nucleic acid. Therefore, a “polynucleotide encoding UGT2B7” refers to a nucleic acid segment that contains UGT2B7 coding sequences, yet is isolated away from, or purified and free of, total genomic DNA and proteins. When the present application refers to the function or activity of a UGT2B7-encoding polynucleotide or nucleic acid, it is meant that the polynucleotide encodes a molecule that has the ability to glucuronidate a substrate, such as epirubicin.

The term “cDNA” is intended to refer to DNA prepared using RNA as a template. The advantage of using a cDNA, as opposed to genomic DNA or an RNA transcript is stability and the ability to manipulate the sequence using recombinant DNA technology (See Sambrook, 1989; Ausubel, 1996). There may be times when the full or partial genomic sequence is preferred. Alternatively, cDNAs may be advantageous because it represents coding regions of a polypeptide and eliminates introns and other regulatory regions.

It also is contemplated that a given UGT2B7-encoding nucleic acid or UGT2B7 gene from a given cell may be represented by natural variants or strains that have slightly different nucleic acid sequences but, nonetheless, encode a UGT2B7 polypeptide; a human UGTB7 polypeptide is a preferred embodiment. Consequently, the present invention also encompasses derivatives of UGT2B7 with minimal amino acid changes, but that possess the same activity.

The term “gene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants. The nucleic acid molecule encoding UGT2B7 or a UGT2B7 modulator, or a UGT2B7 gene or a UGT2B7 modulator gene, may comprise a contiguous nucleic acid sequence of the following lengths: at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 10100, 10200, 10300, 10400, 10500, 10600, 10700, 10800, 10900, 11000, 11100, 11200, 11300, 11400, 11500, 11600, 11700, 11800, 11900, 12000 or more nucleotides, nucleosides, or base pairs. Such sequences may be identical or complementary to SEQ ID NO:1 (UGT2B7 cDNA and promoter sequence), or SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, and/or SEQ ID NO:78 (SEQ ID NOS:3-78) (primers to amplify or sequence all or part of SEQ ID NO:1 or the UGT2B7 gene).

In some embodiments, genetic polymorphisms in UGT2B7 are relevant. As used herein, a “single nucleotide polymorphism” (SNP) refers to an addition, deletion, or substitution of a single nucleotide at a site in a nucleic acid molecule; it reflects the occurrence of genetically determined variant forms of a nucleic acid sequence at a frequency where the rarest could not be maintained by recurrent mutation alone. In some instances, a polymorphism in a sequence results in a change that affects the activity, expression, or stability of a transcript or polypeptide encoded by the sequence. Thus, in some embodiments of the present invention, a polymorphism in a UGT2B7 gene results in a change in effective UGT2B7 enzyme activity or the level of UGT2B7 protein or transcript expression.

“Isolated substantially away from other coding sequences” means that the gene of interest forms part of the coding region of the nucleic acid segment, and that the segment does not contain large portions of naturally-occurring coding nucleic acid, such as large chromosomal fragments or other functional genes or cDNA coding regions. Of course, this refers to the nucleic acid segment as originally isolated, and does not exclude genes or coding regions later added to the segment by human manipulation.

In particular embodiments, the invention concerns isolated DNA segments and recombinant vectors incorporating DNA sequences that encode a UGT2B7 protein, polypeptide or peptide that includes within its amino acid sequence a contiguous amino acid sequence in accordance with, or essentially as set forth in, SEQ ID NO:2, corresponding to the UGT2B7 designated “human UGT2B7.”

The term “a sequence essentially as set forth in SEQ ID NO:2” means that the sequence substantially corresponds to a portion of SEQ ID NO:2 and has relatively few amino acids that are not identical to, or a biologically functional equivalent of, the amino acids of SEQ ID NO:2.

The term “biologically functional equivalent” is well understood in the art and is further defined in detail herein. Accordingly, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of amino acids that are identical or functionally equivalent to the amino acids of SEQ ID NO:2 will be sequences that are “essentially as set forth in SEQ ID NO:2” provided the biological activity of the protein is maintained. In particular embodiments, the biological activity of a UGT2B7 protein, polypeptide or peptide, or a biologically functional equivalent, comprises catalyzing the glucuronidation of a substrate such as epirubicin. In certain other embodiments, the invention concerns isolated DNA segments and recombinant vectors that include within their sequence a nucleic acid sequence essentially as set forth in SEQ ID NO:1. The term “essentially as set forth in SEQ ID NO:1” is used in the same sense as described above and means that the nucleic acid sequence substantially corresponds to a portion of SEQ ID NO:1 and has relatively few codons that are not identical, or functionally equivalent, to the codons of SEQ ID NO:1. Again, DNA segments that encode proteins, polypeptide or peptides exhibiting UGT2B7 activity will be most preferred.

In particular embodiments, the invention concerns isolated nucleic acid segments and recombinant vectors incorporating DNA sequences that encode UGT2B7 polypeptides or peptides that include within its amino acid sequence a contiguous amino acid sequence in accordance with, or essentially corresponding to UGT2B7 polypeptides.

The nucleic acid segments used in the present invention, regardless of the length of the coding sequence itself, may be combined with other DNA or RNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

It is contemplated that the nucleic acid constructs of the present invention may encode UGT2B7 or UGT2B7 modulators. A “heterologous” sequence refers to a sequence that is foreign or exogenous to the remaining sequence. A heterologous gene refers to a gene that is not found in nature adjacent to the sequences with which it is now placed.

In a non-limiting example, one or more nucleic acid constructs may be prepared that include a contiguous stretch of nucleotides identical to or complementary to all or part of a UGT2B7 gene. A nucleic acid construct may comprise at least 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 20,000, 30,000, 50,000, 100,000, 250,000, about 500,000, 750,000, to about 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges,” as used herein, means any length or range including or between the quoted values (i.e., all integers including and between such values). Non-limiting examples of intermediate lengths include about 11, about 12, about 13, about 16, about 17, about 18, about 19, etc.; about 21, about 22, about 23, etc.; about 31, about 32, etc.; about 51, about 52, about 53, etc.; about 101, about 102, about 103, etc.; about 151, about 152, about 153, about 97001, about 1,001, about 1002, about 50,001, about 50,002, about 750,001, about 750,002, about 1,000,001, about 1,000,002, etc. Non-limiting examples of intermediate ranges include about 3 to about 32, about 150 to about 500,001, about 3,032 to about 7,145, about 5,000 to about 15,000, about 20,007 to about 1,000,003, etc.

The nucleic acid segments used in the present invention encompass biologically functional equivalent UGT2B7 proteins and peptides. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by human may be introduced through the application of site-directed mutagenesis techniques, e.g., to introduce improvements to the antigenicity of the protein or to test mutants in order to examine DNA binding activity at the molecular level.

Certain embodiments of the present invention concern various nucleic acids, including vectors, promoters, therapeutic nucleic acids, and other nucleic acid elements involved in transformation and expression in cells. In certain aspects, a nucleic acid comprises a wild-type or a mutant nucleic acid. In particular aspects, a nucleic acid encodes for or comprises a transcribed nucleic acid.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length. A “gene” refers to coding sequence of a gene product, as well as introns and the promoter of the gene product. In addition to the UGT2B7 gene, other regulatory regions such as enhancers for UGT2B7 are contemplated as nucleic acids for use with compositions and methods of the claimed invention.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss”, a double stranded nucleic acid by the prefix “ds”, and a triple stranded nucleic acid by the prefix “ts.”

In particular aspects, a nucleic acid encodes a protein, polypeptide, or peptide. In certain embodiments, the present invention concerns novel compositions comprising at least one proteinaceous molecule. As used herein, a “proteinaceous molecule,” “proteinaceous composition,” “proteinaceous compound,” “proteinaceous chain,” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

1. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 1989, incorporated herein by reference).

2. Purification of Nucleic Acids

A nucleic acid may be purified on polyacrylamide gels, cesium chloride centrifugation gradients, or by any other means known to one of ordinary skill in the art (see for example, Sambrook et al., 1989, incorporated herein by reference). In preferred aspects, a nucleic acid is a pharmacologically acceptable nucleic acid. Pharmacologically acceptable compositions are known to those of skill in the art, and are described herein.

In certain aspect, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

3. Nucleic Acid Segments

In certain embodiments, the nucleic acid is a nucleic acid segment. As used herein, the term “nucleic acid segment,” are fragments of a nucleic acid, such as, for a non-limiting example, those that encode only part of a peptide or polypeptide sequence. Thus, a “nucleic acid segment” may comprise any part of a gene sequence, including from about 2 nucleotides to the full length of a peptide or polypeptide encoding region.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:

n to n+y

where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

4. Nucleic Acid Complements

The present invention also encompasses a nucleic acid that is complementary to a nucleic acid. A nucleic acid is “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule. In preferred embodiments, a complement is an antisense nucleic acid used to reduce expression (e.g., translation) of a RNA transcript in vivo.

As used herein, the term “complementary” or “complement(s)” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. However, in some antisense embodiments, completely complementary nucleic acids are preferred.

5. Vectors Encoding UGT2B7

The present invention encompasses the use of vectors to encode for UGT2B7 and candidate modulators of UGT2B7. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., 1989 and Ausubel et al., 1996, both incorporated herein by reference.

The term “expression vector” or “expression construct” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

a. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202, 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the nucleic acid segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or exogenous, for example, a non-UGT2B7 promoter with respect to UGT2B7 encoding sequence. In some examples, a prokaryotic promoter is employed for use with in vitro transcription of a desired sequence. Prokaryotic promoters for use with many commercially available systems include T7, T3, and Sp6.

Table 2 lists several elements/promoters that may be employed, in the context of the present invention, to regulate the expression of a gene. This list is not intended to be exhaustive of all the possible elements involved in the promotion of expression but, merely, to be exemplary thereof. Table 3 provides examples of inducible elements, which are regions of a nucleic acid sequence that can be activated in response to a specific stimulus.

TABLE 2
Promoter and/or Enhancer

| Promoter/Enhancer | References |
|---|---|
| Immunoglobulin Heavy Chain | Banerji et al., 1983; Gilles et al., 1983; Grosschedl |
| | et al., 1985; Atchinson et al., 1986, 1987; Imler |
| | et al., 1987; Weinberger et al., 1984; Kiledjian |
| | et al., 1988; Porton et al.; 1990 |
| Immunoglobulin Light Chain | Queen et al., 1983; Picard et al., 1984 |
| T-Cell Receptor | Luria et al., 1987; Winoto et al., 1989; Redondo |
| | et al.; 1990 |
| HLA DQ α and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukm-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson |
| | et al., 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr et al., 1986; |
| | Imbra et al., 1986; Kadesch et al., 1986; Wang et |
| | al., 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen |
| | et al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Chol |
| | et al., 1988; Reisman et al., 1989 |
| Papilloma Virus | Campo et al., 1983; Lusky et al., 1983; Spandidos |
| | and/or Wilkie, 1983; Spalholz et al., 1985; Lusky |
| | et al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immunodeficiency Virus | Muesing et al., 1987; Hauber et al., 1988; |
| | Jakobovits et al., 1988; Feng et al., 1988; Takebe |
| | et al., 1988; Rosen et al., 1988; Berkhout et al., |
| | 1989; Laspia et al., 1989; Sharp et al., 1989; |
| | Braddock et al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

TABLE 3
Inducible Elements

| Element | Inducer | References |
|---|---|---|
| MT II | Phorbol Ester | Palmiter et al., 1982; |
| | (TFA) Heavy | Haslinger et al., 1985; |
| | metals | Searle et al., 1985; Stuart |
| | | et al., 1985; Imagawa |
| | | et al., 1987, Karin et al., |
| | | 1987; Angel et al., 1987b; |
| | | McNeall et al., 1989 |
| MMTV (mouse | Glucocorticoids | Huang et al., 1981; Lee |
| mammary tumor | | et al., 1981; Majors et al., |
| virus) | | 1983; Chandler et al., |
| | | 1983; Lee et al., 1984; |
| | | Ponta et al., 1985; Sakai |
| | | et al., 1988 |
| β-Interferon | poly(rI)x | Tavernier et al., 1983 |
| | poly(rc) | |
| Adenovirus 5 E2 | E1A | Imperiale et al., 1984 |
| Collagenase | Phorbol Ester | Angel et al., 1987a |
| | (TPA) | |
| Stromelysin | Phorbol Ester | Angel et al., 1987b |
| | (TPA) | |
| SV40 | Phorbol Ester | Angel et al., 1987b |
| | (TPA) | |
| Murine MX Gene | Interferon, | Hug et al., 1988 |
| | Newcastle | |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb | | |
| HSP70 | E1A, SV40 Large | Taylor et al., 1989, 1990a, |
| | T Antigen | 1990b |
| Proliferin | Phorbol Ester- | Mordacq et al., 1989 |
| | TPA | |
| Tumor Necrosis Factor | PMA | Hensel et al., 1989 |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Examples of such regions include the human LIMEK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), DIA dopamine receptor gene (Lee, et al., 1997), insulin-like growth factor II (Wu et al., 1997), human platelet endothelial cell adhesion molecule-1 (Almendro et al., 1996).

b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

C. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, herein incorporated by reference.)

e. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

f. Polyadenylation Signals

For expression, particularly eukaryotic expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

g. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

h. Selectable and Screenable Markers

In certain embodiments of the invention, the cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

6. Host Cells

As used herein, the terms “cell,” “cell line,” and “cell culture” may be used interchangeably. All of these terms also include their progeny, which refers to any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. A “recombinant host cell” refers to a host cell that carries a recombinant nucleic acid, i.e. a nucleic acid that has been manipulated in vitro or that is a replicated copy of a nucleic acid that has been so manipulated.

A host cell may be derived from prokaryotes or eukaryotes, depending upon whether the desired result is replication of the vector, expression of part or all of the vector-encoded nucleic acid sequences, or production of infectious viral particles. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and Solopack™ Gold Cells (Strategene®, La Jolla). Alternatively, bacterial cells such as E. coli LE392 could be used as host cells for phage viruses.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either an eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

7. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MaxBac® 2.0 from Invitrogen® and BacPack™ Baculovirus Expression System from Clontech®.

Other examples of expression systems include Stratagene®'s Complete Control™ Inducible Mammalian Expression System, which involves a synthetic ecdysone-inducible receptor, or its pET Expression System, an E. coli expression system. Another example of an inducible expression system is available from Invitrogen®, which carries the T-Rex™ (tetracycline-regulated expression) System, an inducible mammalian expression system that uses the full-length CMV promoter. The Tet-On™ and Tet-Off™ systems from Clontech® can be used to regulate expression in a mammalian host using tetracycline or its derivatives. The implementation of these systems is described in Gossen et al., 1992 and Gossen et al., 1995, and U.S. Pat. No. 5,650,298, all of which are incorporated by reference.

Invitrogen® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

8. Viral Vectors

There are a number of ways in which expression vectors may be introduced into cells. In certain embodiments of the invention, the expression vector comprises a virus or engineered vector derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material but can be readily introduced in a variety of cell lines and laboratory animals (Nicolas and Rubenstein, 1988; Temin, 1986).

The retroviruses are a group of single-stranded RNA viruses characterized by an ability to convert their RNA to double-stranded DNA in infected cells; they can also be used as vectors. Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

9. Nucleic Acid Detection

In some embodiments the invention concerns identifying polymorphisms in UGT2B7, correlating genotype to phenotype, wherein the phenotype is lowered UGT2B7 activity or expression, and then identifying such polymorphisms in patients who have or will be given epirubicin. Thus, the present invention involves assays for identifying polymorphisms and other nucleic acid detection methods. Nucleic acids, therefore, have utility as probes or primers for embodiments involving nucleic acid hybridization. They may be used in diagnostic or screening methods of the present invention. Detection of nucleic acids encoding UGT2B7, as well as nucleic acids involved in the expression or stability of UGT2B7 polypeptides or transcripts, are encompassed by the invention.

The following tables provide information regarding UGT2B7 sequences and primers that may be employed in any of the methods described herein. Some of this information was obtained from WO 00/06776.

Table 4 provides primers that can be used to amplify UGT2B7 genomic or cDNA sequences by polymerase chain reaction, which is known to those of ordinary skill, and which is described herein.

TABLE 4

PCR Primers for UGT2B7 Amplification

| | | | |
|---|---|---|---|
| | | SEQ | |
| | Direction | ID | |
| Region | (and name) | NO | Primer Sequence 5′→3′ |

| | | | |
|---|---|---|---|
| UGT2B7 | F (PF) | 3 | GTGTCAATGGACTGCAGAAC |
| Promoter | | | |
| | R (PR) | 4 | CCTTTCCACAATTCCCAGAG |
| UTGT2B7 | F (1FA) | 5 | CTTGGCTAATTTATCTTTGG |
| Exon 1 | | | |
| | R (1RA) | 6 | CCCACTACCCTGACTTTAT |
| | F | 7 | GGACATAACCATGAGAAATG |
| | R | 8 | AGCTCTGCTTCAAAGACAC |
| UTGT2B7 | F(2FA) | 9 | TGTCCGTATGCTACTATTGAA |
| Exon 2 | | | |
| | R | 10 | TGTGCTAATCCCTTTGTAAAT |
| | F | 11 | TTTTTTTTTCTATTCCTGTCAG |
| | R | 12 | CTTTACCCCACCCATT |
| | R (2RD) | 72 | GTTTGGCAGGTTTGCAGTGG |
| UGT2B7 | F (3F) | 73 | GAAGCAAATTCTTTCTTCACAG |
| Exon 3 | | | |
| | R (3R) | 74 | ACCAGTAAGGCACTTCATCTT |
| UTGT2B7 | F(4FA) | 13 | CCCTTGATCTCATTCCTACT |
| Exon 4 | | | |
| | R | 14 | AACTGGCTATTCTTTAGATGTATG |
| | F | 15 | CATTCCTACTCTTTATACAGTTCTC |
| | R | 16 | CCCCCGATTCAGACTAT |
| | R (4RC) | 75 | CGATTCAGACTATAAAGAATGT |
| UTGT2B7 | F | 17 | CCCTTGATCTCATTCCTACT |
| Exon 5 | | | |
| | R | 18 | AACTGGCTATTCTTTAGATG TATG |
| | F | 19 | CCTCCGAAGTCTGAAAC |
| | R | 20 | TATAAAAAAGGATGAAACTCACAC |
| | F (5FB) | 76 | TCCTCCGAAGTCTGAAAC |
| | R (5RB(2)) | 77 | CCACCTAGTGAAAAATATTGTTC |
| UTGT2B7 | F | 21 | CAAGCCCCCAAGTTATGT |
| Exon 6 | | | |
| | R | 22 | CAGTAGGATCCGCGATATAA |
| | F(6FB) | 23 | TCTGAGGGGTTTTGTCTGTA |
| | R(6RB) | 78 | ATCACAATCTTTCTTGCTGGA |
| | R | 24 | CCGCGATATAAGTTCAACAA |

“F” means forward; “R” means reverse.

Table 5 below provides information about primers that can be used to sequence UGT2B7 or UGT2B7-encoding nucleic acid molecules. Standard sequencing protocols can be practiced by one of ordinary skill in the art, and are described herein.

TABLE 5

Sequencing Primers UGT2B7

| | | | |
|---|---|---|---|
| | | SEQ | |
| | | ID | |
| P. No. | F/R | NO | Primer Sequence |

| | | | |
|---|---|---|---|
| 1,2 | F | 25 | GGACATAACCATGAGAAATG |
| | R | 26 | TTAAGAGCGGATGAGTTGT |
| 3,4 | F | 27 | TCATCATGCAACAGATTAAG |
| | R | 28 | CACTACAGGGAAAAATAGCA |
| 5 | F | 29 | ACCCTTTGTGTACAGTCTCA |
| | R | 30 | AGCTCTGCTTCAAAGACAC |
| 6,7 | F | 31 | TTGCCTACATTATTCTAACCC |
| | R | 32 | CTTTACCCCACCCATTT |
| 8,9 | F | 33 | CATTCCTACTCTTTATACAGTTCTC |
| | R | 34 | CCCCCGATTCAGACTAT |
| 10 | F | 35 | CATTCCTACTCTTTATACAGTTCTC |
| | R | 36 | CCCCCGATTCAGACTAT |
| 11,12 | F | 37 | TCCTCCGAAGTCTGAAAC |
| | R | 38 | TATAAAAAGGATGAAACTCACAC |
| 13 | F | 39 | TCTGAGGGGTTTTGTCTGTA |
| | R | 40 | TTTTTTGTCTCAGGAAGAAAGA |
| 14 | F | 41 | AAAAAAAGAAAAAAAAATCTTTTC |
| | R | 42 | CCGCGATATAAGTTCAACAA |
| | primer | 71 | TCTGAGCATGTGGATGGCAA |
| | extension) | | |

F/R refers to forward or reverse primers

Table 6 provides sequence information about polymorphisms identified in the coding and noncoding regions of UGT2B7. The changes and position in the sequence, and any consequent amino acid change, is provided in the table.

TABLE 6
Summary of Known Sequence Polymorphisms UGT2B7

| N | Region | Nt Change | AA Change | SEQ ID NO | Sequence |
|---|---|---|---|---|---|
| 1 | Upstream | G −2 A | | 43 | TGCATTGCACCAGGATGTCTGT |
| | | | | 44 | TGCATTGCACCAAGATGTCTGT |
| 2 | Exon 1 | T +137 C | Leu +46 Phe | 45 | TCCTGGATGAGCTTATTCAGAGA |
| | | | | 46 | TCCTGGATGAGCCTATTCAGAGA |
| 3 | Exon 1 | A +321 T | | 47 | CATTTTGGTTATATTTTTCAC |
| | | | | 48 | CATTTTGGTTTTATTTTTCAC |
| 4 | Exon 1 | A +372 G | | 49 | CATAACTAGAAAGTTCTGTAA |
| | | | | 50 | CATAACTAGGAAGTTCTGTAA |
| 5 | Exon 1 | C +536 T | Thr +179 Ile | 51 | CCTGGCTACACTTTTGAAAA |
| | | | | 52 | CCTGGCTACATTTTTGAAAA |
| 6 | Exon 2 | A +735 G | | 53 | GAAGACCCACTACATTATCTG |
| | | | | 54 | GAAGACCCACTACGTTATCTG |
| 7 | Exon 2 | AT +801-802 TC | His +268 Tyr | 55 | AATTTTCAGTTTCCATATCCACTCTT |
| | | | | 56 | AATTTTCAGTTTCCTCATCCACTCTT |
| 8 | Exon 4 | C +1059 G | | 57 | TAGGTCTCAATACTCGGCTC TA |
| | | | | 58 | TAGGTCTCAATACTCGGCTGTA |
| 9 | Exon 4 | C +1062 T | | 59 | TACAAGTGGATACCCCAGA |
| | | | | 60 | TATAAGTGGATACCCCAGA |
| 10 | Intron 4 | A +154 del | | 61 | GGGAGAAAGAATACATTATAATTTTT |
| | | | | 62 | GGGAGAAAGAATACTTATAATTTTT |
| 11 | Exon 5 | C +1191 T | | 63 | TTCCATTGTTTGCCGATCAAC |
| | | | | 64 | TTCCATTGTTTGCTGATCAAC |
| 12 | Exon 5 | A +1288 C | Lys +430 Gln | 65 | GAATGCATTGAAGAGAGTAAT |
| | | | | 66 | GAATGCATTGCAGAGAGTAAT |
| 13 | Exon 6 | A +1506 G | | 67 | CTGGTCTGTGTGGCAACTGTGA |
| | | | | 68 | CTGGTCTGTGTGGCGACTGTGA |
| 14 | 3′ UTR | C +1838 A | | 69 | TAAGATAAAGCCTTATGAG |
| | | | | 70 | TAAGATAAAGACTTATGAG |

**Nt change refers to nucleotide change; AA change refers to resulting amino acid change, where the first methionine in the polypeptide is designated +1.

General methods of nucleic acid detection methods are provided below, followed by specific examples employed for the identification of polymorphisms, including single nucleotide polymorphisms (SNPs).

a. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, site-directed mutagenesis, it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

b. Amplification of Nucleic Acids

Nucleic acids used as a template for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to SEQ ID NO:1, SEQ ID NOS:3-78 or any other SEQ ID NO if appropriate, are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA) (described in further detail below), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

C. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 1989). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

d. Other Assays

Other methods for genetic screening may be used within the scope of the present invention, for example, to detect mutations in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR™ (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substitution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

e. Specific Examples of SNP Screening Methods

Spontaneous mutations that arise during the course of evolution in the genomes of organisms are often not immediately transmitted throughout all of the members of the species, thereby creating polymorphic alleles that co-exist in the species populations. Often polymorphisms are the cause of genetic diseases. Several classes of polymorphisms have been identified. For example, variable nucleotide type polymorphisms (VNTRs), arise from spontaneous tandem duplications of di- or trinucleotide repeated motifs of nucleotides. If such variations alter the lengths of DNA fragments generated by restriction endonuclease cleavage, the variations are referred to as restriction fragment length polymorphisms (RFLPs). RFLPs are been widely used in human and animal genetic analyses.

Another class of polymorphisms are generated by the replacement of a single nucleotide. Such single nucleotide polymorphisms (SNPs) rarely result in changes in a restriction endonuclease site. Thus, SNPs are rarely detectable restriction fragment length analysis. SNPs are the most common genetic variations and occur once every 100 to 300 bases and several SNP mutations have been found that affect a single nucleotide in a protein-encoding gene in a manner sufficient to actually cause a genetic disease. SNP diseases are exemplified by hemophilia, sickle-cell anemia, hereditary hemochromatosis, late-onset Alzheimer disease etc.

In context of the present invention, polymorphic mutations that affect the activity and/or levels of the UGT2B7 gene products, which are responsible for the glucuronidation of epirubicin and other chemotherapeutic and xenobiotic agents, will be determined by a series of screening methods. One set of screening methods is aimed at identifying SNPs that affect the activity and/or level of the UGT2B7 gene products in in vitro assays. The other set of screening methods will then be performed to screen an individual for the occurrence of the SNPs identified above. To do this, a sample (such as blood or other bodily fluid or tissue sample) will be taken from a patient for genotype analysis. The presence or absence of SNPs will determine the ability of the screened individuals to metabolize epirubicin and other chemotherapeutic agents that are metabolized by the UGTB27 gene products. According to methods provided by the invention, these results will be used to adjust and/or alter the dose of epirubicin or other agent administered to an individual in order to reduce drug side effects.

SNPs can be the result of deletions, point mutations and insertions and in general any single base alteration, whatever the cause, can result in a SNP. The greater frequency of SNPs means that they can be more readily identified than the other classes of polymorphisms. The greater uniformity of their distribution permits the identification of SNPs “nearer” to a particular trait of interest. The combined effect of these two attributes makes SNPs extremely valuable. For example, if a particular trait (e.g., inability to efficiently metabolize epirubicin) reflects a mutation at a particular locus, then any polymorphism that is linked to the particular locus can be used to predict the probability that an individual will be exhibit that trait.

Several methods have been developed to screen polymorphisms and some examples are listed below. SNPs relating to glucuronidation of chemotherapeutic agents can be characterized by the use of any of these methods or suitable modification thereof. Such methods include the direct or indirect sequencing of the site, the use of restriction enzymes where the respective alleles of the site create or destroy a restriction site, the use of allele-specific hybridization probes, the use of antibodies that are specific for the proteins encoded by the different alleles of the polymorphism, or any other biochemical interpretation.

i) DNA Sequencing

The most commonly used method of characterizing a polymorphism is direct DNA sequencing of the genetic locus that flanks and includes the polymorphism. Such analysis can be accomplished using either the “dideoxy-mediated chain termination method,” also known as the “Sanger Method” (Sanger, F., et al., 1975) or the “chemical degradation method,” also known as the “Maxam-Gilbert method” (Maxam, A. M., et al., 1977). Sequencing in combination with genomic sequence-specific amplification technologies, such as the polymerase chain reaction may be utilized to facilitate the recovery of the desired genes (Mullis, K. et al., 1986; European Patent Appln. 50,424; European Patent Appln. 84,796, European Patent Application 258,017, European Patent Appln. 237,362; European Patent Appln. 201,184; U.S. Pat. Nos. 4,683,202; 4,582,788; and 4,683,194), all of the above incorporated herein by reference.

ii) Exonuclease Resistance

Other methods that can be employed to determine the identity of a nucleotide present at a polymorphic site utilize a specialized exonuclease-resistant nucleotide derivative (U.S. Pat. No. 4,656,127). A primer complementary to an allelic sequence immediately 3′-to the polymorphic site is hybridized to the DNA under investigation. If the polymorphic site on the DNA contains a nucleotide that is complementary to the particular exonucleotide-resistant nucleotide derivative present, then that derivative will be incorporated by a polymerase onto the end of the hybridized primer. Such incorporation makes the primer resistant to exonuclease cleavage and thereby permits its detection. As the identity of the exonucleotide-resistant derivative is known one can determine the specific nucleotide present in the polymorphic site of the DNA.

iii) Microsequencing Methods

Several other primer-guided nucleotide incorporation procedures for assaying polymorphic sites in DNA have been described (Komher, J. S. et al., 1989; Sokolov, B. P., 1990; Syvanen 1990; Kuppuswamy et al., 1991; Prezant et al., 1992; Ugozzoll, L. et al., 1992; Nyren et al., 1993). These methods rely on the incorporation of labeled deoxynucleotides to discriminate between bases at a polymorphic site. As the signal is proportional to the number of deoxynucleotides incorporated, polymorphisms that occur in runs of the same nucleotide result in a signal that is proportional to the length of the run (Syvanen et al.,1993).

iv) Extension in Solution

French Patent 2,650,840 and PCT Application No. WO91/02087 discuss a solution-based method for determining the identity of the nucleotide of a polymorphic site. According to these methods, a primer, complementary to allelic sequences immediately 3′-to a polymorphic site is used. The identity of the nucleotide of that site is determined using labeled dideoxynucleotide derivatives which are incorporated at the end of the primer if complementary to the nucleotide of the polymorphic site.

v) Genetic Bit™ Analysis or Solid-Phase Extension

PCT Appln. No. 92/15712 describes a method that uses mixtures of labeled terminators and a primer that is complementary to the sequence 3′ to a polymorphic site. The labeled terminator that is incorporated is complementary to the nucleotide present in the polymorphic site of the target molecule being evaluated and is thus identified. Here the primer or the target molecule is immobilized to a solid phase.

vi) Oligonucleotide Ligation Assay (OLA)

This is another solid phase method that uses different methodology (Landegren et al., 1988). Two oligonucleotides, capable of hybridizing to abutting sequences of a single strand of a target DNA are used. One of these oligonucleotides is biotinylated while the other is detectably labeled. If the precise complementary sequence is found in a target molecule, the oligonucleotides will hybridize such that their termini abut, and create a ligation substrate. Ligation permits the recovery of the labeled oligonucleotide by using avidin. Other nucleic acid detection assays, based on this method, combined with PCR™ are also described (Nickerson et al., 1990). Here PCR is used to achieve the exponential amplification of target DNA, which is then detected using the OLA.

vii) Ligase/Polymerase-Mediated Genetic Bit

Analysis

U.S. Pat. No. 5,952,174 describes a method that also involves two primers capable of hybridizing to abutting sequences of a target molecule. The hybridized product is formed on a solid support to which the target is immobilized. Here the hybridization occurs such that the primers are separated from one another by a space of a single nucleotide. Incubating this hybridized product in the presence of a polymerase, a ligase, and a nucleoside triphosphate mixture containing at least one deoxynucleoside triphosphate allows the ligation of any pair of abutting hybridized oligonucleotides. Addition of a ligase results in two events required to generate a signal, extension and ligation. This provides a higher specificity and lower “noise” than methods using either extension or ligation alone and unlike the polymerase-based assays, this method enhances the specificity of the polymerase step by combining it with a second hybridization and a ligation step for a signal to be attached to the solid phase.

viii) Other Methods to Detect SNPs

Several other specific methods for SNP detection and identification are presented below and may be used as such or with suitable modifications in conjunction with identifying polymorphisms of the UGT2B7 genes in the present invention. Several other methods are also described on the SNP web site of the NCBI at http://www.ncbi.nlm.nih.gov/SNP, incorporated herein by reference.

The VDA-assay utilizes PCR amplification of genomic segments by long PCR methods using TaKaRa LA Taq reagents and other standard reaction conditions. The long amplification can amplify DNA sizes of about 2,000-12,000 bp. Hybridization of products to variant detector array (VDA) can be performed by a Affymetrix High Throughput Screening Center and analyzed with computerized software.

A method called Chip Assay uses PCR amplification of genomic segments by standard or long PCR protocols. Hybridization products are analyzed by VDA, Halushka et al., 1999, incorporated herein by reference. SNPs are generally classified as “Certain” or “Likely” based on computer analysis of hybridization patterns. By comparison to alternative detection methods such as nucleotide sequencing, “Certain” SNPs have been confirmed 100% of the time; and “Likely” SNPs have been confirmed 73% of the time by this method.

Other methods simply involve PCR amplification following digestion with the relevant restriction enzyme. Yet others involve sequencing of purified PCR products from known genomic regions.

In yet another method, individual exons or overlapping fragments of large exons are PCR-amplified. Primers are designed from published or database sequences and PCR-amplification of genomic DNA is performed using the following conditions: 200 ng DNA template, 0.5 μM each primer, 80 μM each of dCTP, dATP, dTTP and dGTP, 5% formamide, 1.5 mM MgCl2, 0.5 U of Taq polymerase and 0.1 volume of the Taq buffer. Thermal cycling is performed and resulting PCR-products are analyzed by PCR-single strand conformation polymorphism (PCR-SSCP) analysis, under a variety of conditions, e.g, 5 or 10% polyacrylamide gel with 15% urea, with or without 5% glycerol. Electrophoresis is performed overnight. PCR-products that show mobility shifts are reamplified and sequenced to identify nucleotide variation.

In a method called CGAP-GAI (DEMIGLACE), sequence and alignment data (from a PHRAP.ace file), quality scores for the sequence base calls (from PHRED quality files), distance information (from PHYLIP dnadist and neighbour programs) and base-calling data (from PHRED ‘-d’ switch) are loaded into memory. Sequences are aligned and examined for each vertical chunk (‘slice’) of the resulting assembly for disagreement. Any such slice is considered a candidate SNP (DEMIGLACE). A number of filters are used by DEMIGLACE to eliminate slices that are not likely to represent true polymorphisms. These include filters that: (i) exclude sequences in any given slice from SNP consideration where neighboring sequence quality scores drop 40% or more; (ii) exclude calls in which peak amplitude is below the fifteenth percentile of all base calls for that nucleotide type; (iii) disqualify regions of a sequence having a high number of disagreements with the consensus from participating in SNP calculations; (iv) removed from consideration any base call with an alternative call in which the peak takes up 25% or more of the area of the called peak; (v) exclude variations that occur in only one read direction. PHRED quality scores were converted into probability-of-error values for each nucleotide in the slice. Standard Baysian methods are used to calculate the posterior probability that there is evidence of nucleotide heterogeneity at a given location.

In a method called CU-RDF (RESEQ), PCR amplification is performed from DNA isolated from blood using specific primers for each SNP, and after typical cleanup protocols to remove unused primers and free nucleotides, direct sequencing using the same or nested primers.

In a method called DEBNICK (METHOD-B), a comparative analysis of clustered EST sequencesis performed and confirmed by fluorescent-based DNA sequencing. In a related method, called DEBNICK (METHOD-C), comparative analysis of clustered EST sequences with phred quality >20 at the site of the mismatch, average phred quality >=20 over 5 bases 5′-FLANK and 3′ to the SNP, no mismatches in 5 bases 5′ and 3′ to the SNP, at least two occurrences of each allele is performed and confirmed by examining traces.

In a method identified by ERO (RESEQ), new primers sets are designed for electronically published STSs and used to amplify DNA from 10 different mouse strains. The amplification product from each strain is then gel purified and sequenced using a standard dideoxy, cycle sequencing technique with ³³P-labeled terminators. All the ddATP terminated reactions are then loaded in adjacent lanes of a sequencing gel followed by all of the ddGTP reactions and so on. SNPs are identified by visually scanning the radiographs.

In another method identified as ERO (RESEQ-HT), new primers sets are designed for electronically published murine DNA sequences and used to amplify DNA from 10 different mouse strains. The amplification product from each strain is prepared for sequencing by treating with Exonuclease I and Shrimp Alkaline Phosphatase. Sequencing is performed using ABI Prism Big Dye Terminator Ready Reaction Kit (Perkin-Elmer) and sequence samples are run on the 3700 DNA Analyzer (96 Capillary Sequencer).

FGU-CBT (SCA2-SNP) identifies a method where the region containing the SNP is PCR amplified using the primers SCA2-FP3 (5′ CTCCGCCTCAGACTGTTTTGGTAG 3′) and SCA2-RP3 (5′ GTGGCCGAGGACGAGGAGAC 3′). Approximately 100 ng of genomic DNA is amplified in a 50 ml reaction volume containing a final concentration of 5 mM Tris, 25 mM KCl, 0.75 mM MgCl2, 0.05% gelatin, 20 pmol of each primer and 0.5 U of Taq DNA polymerase. Samples are denatured, annealed and extended and the PCR product is purified from band cut out of the agarose gel using, for example, the QIAquick gel extraction kit (Qiagen) and is sequenced using dye terminator chemistry on an ABI Prism 377 automated DNA sequencer with the PCR primers.

In a method identified as JBLACK (SEQ/RESTRICT), two independent PCR reactions are performed with genomic DNA. Products from the first reaction are analyzed by sequencing, indicating a unique FspI restriction site. The mutation is confirmed in the product of the second PCR reaction by digesting with Fsp I.

In a method described as KWOK(1), SNPs are identified by comparing high quality genomic sequence data from four randomly chosen individuals by direct DNA sequencing of PCR products with dye-terminator chemistry (see Kwok et al., 1996). In a related method identified as KWOK (2) SNPs) are identified by comparing high quality genomic sequence data from overlapping large-insert clones such as bacterial artificial chromosomes (BACs) or P1-based artificial chromosomes (PACs). An STS containing this SNP is then developed and the existence of the SNP in various populations is confirmed by pooled DNA sequencing (see Taillon-Miller et al., 1998). In another similar method called KWOK(3), SNPs are identified by comparing high quality genomic sequence data from overlapping large-insert clones BACs or PACs. The SNPs found by this approach represent DNA sequence variations between the two donor chromosomes but the allele frequencies in the general population have not yet been determined. In method KWOK(5), SNPs are identified by comparing high quality genomic sequence data from a homozygous DNA sample and one or more pooled DNA samples by direct DNA sequencing of PCR products with dye-terminator chemistry. The STSs used are developed from sequence data found in publicly available databases. Specifically, these STSs are amplified by PCR against a complete hydatidiform mole (CHM) that has been shown to be homozygous at all loci and a pool of DNA samples from 80 CEPH parents (see Taillon-Miller et al., 1999).

In another such method, KWOK (OverlapSnpDetectionWithPolyBayes), SNPs are discovered by automated computer analysis of overlapping regions of large-insert human genomic clone sequences. For data acquisition, clone sequences are obtained directly from large-scale sequencing centers. This is necessary because base quality sequences are not present/available through GenBank. Raw data processing involves analyzed of clone sequences and accompanying base quality information for consistency. Finished (‘base perfect’, error rate lower than 1 in 10,000 bp) sequences with no associated base quality sequences are assigned a uniform base quality value of 40 (1 in 10,000 bp error rate). Draft sequences without base quality values are rejected. Processed sequences are entered into a local database. A version of each sequence with known human repeats masked is also stored. Repeat masking is performed with the program “MASKERAID.” Overlap detection: Putative overlaps are detected with the program “WUBLAST.” Several filtering steps followed in order to eliminate false overlap detection results, i.e. similarities between a pair of clone sequences that arise due to sequence duplication as opposed to true overlap. Total length of overlap, overall percent similarity, number of sequence differences between nucleotides with high base quality value “high-quality mismatches.” Results are also compared to results of restriction fragment mapping of genomic clones at Washington University Genome Sequencing Center, finisher's reports on overlaps, and results of the sequence contig building effort at the NCBI. SNP detection: Overlapping pairs of clone sequence are analyzed for candidate SNP sites with the ‘POLYBAYES’ SNP detection software. Sequence differences between the pair of sequences are scored for the probability of representing true sequence variation as opposed to sequencing error. This process requires the presence of base quality values for both sequences. High-scoring candidates are extracted. The search is restricted to substitution-type single base pair variations. Confidence score of candidate SNP is computed by the POLYBAYES software.

In method identified by KWOK (TaqMan assay), the TaqMan assay is used to determine genotypes for 90 random individuals. In method identified by KYUGEN(Q1), DNA samples of indicated populations are pooled and analyzed by PLACE-SSCP. Peak heights of each allele in the pooled analysis are corrected by those in a heterozygote, and are subsequently used for calculation of allele frequencies. Allele frequencies higher than 10% are reliably quantified by this method. Allele frequency=0 (zero) means that the allele was found among individuals, but the corresponding peak is not seen in the examination of pool. Allele frequency=0-0.1 indicates that minor alleles are detected in the pool but the peaks are too low to reliably quantify.

In yet another method identified as KYUGEN (Method1), PCR products are post-labeled with fluorescent dyes and analyzed by an automated capillary electrophoresis system under SSCP conditions (PLACE-SSCP). Four or more individual DNAs are analyzed with or without two pooled DNA (Japanese pool and CEPH parents pool) in a series of experiments. Alleles are identified by visual inspection. Individual DNAs with different genotypes are sequenced and SNPs identified. Allele frequencies are estimated from peak heights in the pooled samples after correction of signal bias using peak heights in heterozygotes. For the PCR primers are tagged to have 5′-ATT or 5′-GTT at their ends for post-labeling of both strands. Samples of DNA (10 ng/ul) are amplified in reaction mixtures containing the buffer (10 mM Tris-HCl, pH 8.3 or 9.3, 50 mM KCl, 2.0 mM MgCl2), 0.25 μM of each primer, 200 μM of each dNTP, and 0.025 units/μl of Taq DNA polymerase premixed with anti-Taq antibody. The two strands of PCR products are differentially labeled with nucleotides modified with R110 and R6G by an exchange reaction of Klenow fragment of DNA polymerase I. The reaction is stopped by adding EDTA, and unincorporated nucleotides are dephosphorylated by adding calf intestinal alkaline phosphatase. For the SSCP: an aliquot of fluorescently labeled PCR products and TAMRA-labeled internal markers are added to deionized formamide, and denatured. Electrophoresis is performed in a capillary using an ABI Prism 310 Genetic Analyzer. Genescan softwares (P-E Biosystems) are used for data collection and data processing. DNA of individuals (two to eleven) including those who showed different genotypes on SSCP are subjected for direct sequencing using big-dye terminator chemistory, on ABI Prism 310 sequencers. Multiple sequence trace files obtained from ABI Prism 310 are processed and aligned by Phred/Phrap and viewed using Consed viewer. SNPs are identified by PolyPhred software and visual inspection.

In yet another method identified as KYUGEN (Method2), individuals with different genotypes are searched by denaturing HPLC (DHPLC) or PLACE-SSCP (Inazuka et al., 1997) and their sequences are determined to identify SNPs. PCR is performed with primers tagged with 5′-ATT or 5′-GTT at their ends for post-labeling of both strands. DHPLC analysis is carried out using the WAVE DNA fragment analysis system (Transgenomic). PCR products are injected into DNASep column, and separated under the conditions determined using WAVEMaker program (Transgenomic). The two strands of PCR products that are differentially labeled with nucleotides modified with R110 and R6G by an exchange reaction of Klenow fragment of DNA polymerase I. The reaction is stopped by adding EDTA, and unincorporated nucleotides are dephosphorylated by adding calf intestinal alkaline phosphatase. SSCP followed by electrophoresis is performed in a capillary using an ABI Prism 310 Genetic Analyzer. Genescan softwares (P-E Biosystems). DNA of individuals including those who showed different genotypes on DHPLC or SSCP are subjected for direct sequencing using big-dye terminator chemistory, on ABI Prism 310 sequencer. Multiple sequence trace files obtained from ABI Prism 310 are processed and aligned by Phred/Phrap and viewed using Consed viewer. SNPs are identified by PolyPhred software and visual inspection. Trace chromatogram data of EST sequences in Unigene are processed with PHRED. To identify likely SNPs, single base mismatches are reported from multiple sequence alignments produced by the programs PHRAP, BRO and POA for each Unigene cluster. BRO corrected possible misreported EST orientations, while POA identified and analyzed non-linear alignment structures indicative of gene mixing/chimeras that might produce spurious SNPs. Bayesian inference is used to weigh evidence for true polymorphism versus sequencing error, misalignment or ambiguity, misclustering or chimeric EST sequences, assessing data such as raw chromatogram height, sharpness, overlap and spacing; sequencing error rates; context-sensitivity; cDNA library origin, etc.

In method identified as MARSHFIELD(Method-B), overlapping human DNA sequences which contained putative insertion/deletion polymorphisms are identified through searches of public databases. PCR primers which flanked each polymorphic site are selected from the consensus sequences. Primers are used to amplify individual or pooled human genomic DNA. Resulting PCR products are resolved on a denaturing polyacrylamide gel and a PhosphorImager is used to estimate allele frequencies from DNA pools.

10. Methods of Nucleic Acid Transfer

For some methods of the present invention, methods of nucleic acid transfer may be employed. Suitable methods for nucleic acid delivery to effect expression of compositions of the present invention are believed to include virtually any method by which a nucleic acid (e.g., DNA, including viral and nonviral vectors) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

11. Nucleic Acid Arrays

Because the present invention includes kits to implement methods of the invention, the use of arrays or array technology in these kits is specifically contemplated. The term “array” as used herein refers to a systematic arrangement of nucleic acid. For example, a DNA population that is representative of the different alleles of UGT2B7 polymorphisms is divided up into the minimum number of pools in which a desired screening procedure can be utilized to detect a the alleles and which can be distributed into a single multi-well plate. Arrays may be of an aqueous suspension of a DNA population, comprising: a multi-well plate containing a plurality of individual wells, each individual well containing an aqueous suspension of a different content of a DNA population (i.e., different alleles of same polymorphism and/or different polymorphisms, including polymorphisms in complete LD with polymorphism −161). The DNA population may include DNA of a predetermined size. Furthermore, the DNA population in all the wells of the plate may be representative of substantially all the UGT2B7 polymorphisms, as well as polymorohisms in any other gene that is related to dosing of a UGT2B7 glucuronidated substrate. Examples of arrays, their uses, and implementation of them can be found in U.S. Pat. Nos. 6,329,209, 6,329,140, 6,324,479, 6,322,971, 6,316,193, 6,309,823, 5,412,087, 5,445,934, and 5,744,305, which are herein incorporated by reference.

The term a “nucleic acid array” refers to a plurality of target elements, each target element comprising one or more nucleic acid molecules immobilized on one or more solid surfaces to which sample nucleic acids can be hybridized. The nucleic acids of a target element can contain sequence(s) from specific alleles of UGT2B7 polymorphisms. Other target elements will contain, for instance, reference sequences. Target elements of various dimensions can be used in the arrays of the invention. Generally, smaller, target elements are preferred. Typically, a target element will be less than about 1 cm in diameter. Generally element sizes are from 1 μm to about 3 mm, between about 5 μm and about 1 mm. The target elements of the arrays may be arranged on the solid surface at different densities. The target element densities will depend upon a number of factors, such as the nature of the label, the solid support, and the like. One of skill will recognize that each target element may comprise a mixture of nucleic acids of different lengths and sequences. Thus, for example, a target element may contain more than one copy of a nucleic acid, and each copy may be broken into fragments of different lengths. The length and complexity of the nucleic acid fixed onto the target element is not critical to the invention. One of skill can adjust these factors to provide optimum hybridization and signal production for a given hybridization procedure, and to provide the required resolution among different genes or genomic locations. In various embodiments, target element sequences will have a complexity between about 1 kb and about 1 Mb, between about 10 kb to about 500 kb, between about 200 to about 500 kb, and from about 50 kb to about 150 kb.

Microarrays are known in the art and consist of a surface to which probes that correspond in sequence to gene products (e.g., cDNAs, mRNAs, cRNAs, polypeptides, and fragments thereof), can be specifically hybridized or bound at a known position. In one embodiment, the microarray is an array (i.e., a matrix) in which each position represents a discrete binding site for one or both alleles of a UGT2B7 polymorphism and may include alleles from more than one UGT2B7 polymorphism, or at least 1, 2, 3, 4, 5, 6, 7, 8, 9, 10, 11, 12, 13, 14, 15 or more such polymorphisms, including those in complete LD with −161. In a preferred embodiment, the “binding site” (hereinafter, “site”) is a nucleic acid or nucleic acid analogue to which a particular DNA can specifically hybridize. The nucleic acid or analogue of the binding site can be, e.g., a synthetic oligomer, a full-length cDNA, genomic DNA, a less-than full length cDNA, or a gene fragment.

The nucleic acid or analogue are attached to a solid support, which may be made from glass, plastic (e.g., polypropylene, nylon), polyacrylamide, nitrocellulose, or other materials. A preferred method for attaching the nucleic acids to a surface is by printing on glass plates, as is described generally by Schena et al., 1995a. See also DeRisi et al., 1996; Shalon et al., 1996; Schena et al., 1995b. Each of these articles is incorporated by reference in its entirety.

Other methods for making microarrays, e.g., by masking (Maskos et al., 1992), may also be used. In principal, any type of array, for example, dot blots on a nylon hybridization membrane (see Sambrook et al., 1989, which is incorporated in its entirety for all purposes), could be used, although, as will be recognized by those of skill in the art, very small arrays will be preferred because hybridization volumes will be smaller.

It is also contemplated that kits may involve a variety of gene chip formats are described in the art, for example U.S. Pat. Nos. 5,861,242 and 5,578,832 which are expressly incorporated herein by reference. A means for applying the disclosed methods to the construction of such a chip or array would be clear to one of ordinary skill in the art. In brief, the basic structure of a gene chip or array comprises: (1) an excitation source; (2) an array of probes; (3) a sampling element; (4) a detector; and (5) a signal amplification/treatment system. A chip may also include a support for immobilizing the probe.

B. Proteinaceous Compositions

In certain embodiments, the present invention concerns novel compositions or methods comprising at least one proteinaceous molecule. The proteinaceous molecule may be UGT2B7 (SEQ ID NO: 2) or a modulator of UGT2B7, including an inducer of UGT2B7. The proteinaceous molecule may also be used, for example, a UGT2B7 inducer, in a pharmaceutical composition for the delivery of a therapeutic agent, or UGT2B7 may be used as part of a screening assay for UGT2B7 modulators. As used herein, a “proteinaceous molecule,” “proteinaceous composition,” “proteinaceous compound,” “proteinaceous chain,” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

In certain embodiments the size of the at least one proteinaceous molecule may comprise, but is not limited to, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino molecule residues, and any range derivable therein.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

The present application is directed to the function or activity of UGT2B7, which has the ability to catalyze glucuronidation of its substrate. The translated product of SEQ ID NO:1 is provided by SEQ ID NO:2. It is contemplated that the compositions and methods disclosed herein may be utilized to express part or all of SEQ ID NO:2. Determination of which molecules possess this ability may be achieved using functional assays measuring specificity and rate of glucuronidation familiar to those of skill in the art.

1. Protein Purification

It may be desirable to purify UGT2B7 or UGT2B7 modulator polypeptides, heterologous peptides and polypeptides, or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “—fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “—fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (e.g., alter pH, ionic strength, and temperature.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand also should provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

III. Screening for Modulators of the UGT2B7

The present invention further comprises methods for identifying modulators of UGT2B7. A UGT2B7 modulator refers to a compound that is able to increase or reduce effective UGT2B7 amount, expression, transcription, translation, or functional activity. The UGT2B7 modulator may be an agonist (inducer) or antagonist (inhibitor) of UGT2B7. These assays may comprise random screening of large libraries of candidate substances; alternatively, the assays may be used to focus on particular classes of compounds selected with an eye towards structural attributes that are believed to make them more likely to modulate UGT2B7.

By activity, it is meant that one may assay for a measurable effect on UGT2B7 enzyme activity. To identify a UGT2B7 modulator, one generally will determine the activity UGT2B7 in the presence and absence of a candidate substance, wherein a modulator is defined as any substance that alters the amount or activity. For example, a method generally comprises:

(a) providing a candidate modulator;

(b) admixing the candidate modulator with UGT2B7 in the presence of a UGT2B7 substrate under conditions that allow UGT2B7 to glucuronidate the substrate;

(c) measuring the rate or extent of glucuronidation of the substrate in step (b); and

(d) comparing the rate or extent of glucuronidation measured in step (c) with the rate or extent of glucuronidation in the absence of the candidate modulator,

wherein a difference between the measured characteristics indicates that said candidate modulator is, indeed, a modulator of the compound or cell.

Assays may be conducted in cell free systems, in isolated cells, or in organisms including transgenic animals.

It will, of course, be understood that all the screening methods of the present invention are useful in themselves notwithstanding the fact that effective candidates may not be found. The invention provides methods for screening for such candidates, not solely methods of finding them.

A. Modulators

As used herein the term “candidate substance” refers to any molecule that may potentially inhibit or enhance the effective level of UGT2B7 activity or expression. A UGT2B7 inducer refers to a substance that increases the effective level of UGT2B7 activity or expression. A UGT2B7 inhibitor refers to a substance that decreases or reduces the effective level of UGT2B7 activity or expression. It is contemplated that the terms inhibitor and inducer are relative to conditions when the inhibitor or inducer is not present. It is also contemplated that providing UGT2B7 to a cell such that UGT2B7 activity is increased in that cell is an example of UGT2B7 being a UGT2B7 inducer. Alternatively, a UGT2B7 inducer may be transcription factor that increases UGT2B7 transcript levels, which leads to an increase in UGT2B7 expression levels.

The candidate substance may be a protein or fragment thereof, a small molecule, or even a nucleic acid molecule. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are well known to those of skill in the art. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one which exerts its inhibitory or activating effect upstream, downstream or directly on UGT2B7. Regardless of the type of inhibitor or activator identified by the present screening methods, the effect of the inhibition or activator by such a compound results in alteration in overall UGT2B7 enzymatic activity as compared to that observed in the absence of the added candidate substance.

B. In vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule in a specific fashion is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

IV. Pharmaceutical Compositions

Aqueous compositions of the present invention will have an effective amount of a UGT2B7 inducer such that UGT2B7 activity levels are increased in a patient administered the compoision. Such compositions will generally be dissolved or dispersed in a pharmaceutically acceptable carrier or aqueous medium. Other aspects of the invention concern epirubicin administration and dosages, which will be discussed below.

The phrases “pharmaceutically or pharmacologically acceptable” refer to molecular entities and compositions that do not produce an adverse, allergic or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-cancer agents, can also be incorporated into the compositions.

In addition to the compounds formulated for parenteral administration, such as intravenous or intramuscular injection, other pharmaceutically acceptable forms include, e.g., tablets or other solids for oral administration; time release capsules; and any other form currently used, including cremes, lotions, mouthwashes, inhalants and the like.

A. Parenteral Administration

The active compounds will often be formulated for parenteral administration, e.g., formulated for injection via the intravenous, intramuscular, sub-cutaneous, or even intraperitoneal routes. The preparation of an aqueous composition that contains flavopiridol and a second agent as active ingredients will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In certain cases, the therapeutic formulations of the invention could also be prepared in forms suitable for topical administration, such as in cremes and lotions. These forms may be used for treating skin-associated diseases, such as various sarcomas.

Upon formulation, solutions will be administered in a manner compatible with the dosage formulation and in such amount as is therapeutically effective. The formulations are easily administered in a variety of dosage forms, such as the type of injectable solutions described above, with even drug release capsules and the like being employable.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

B. Oral Administration

In certain embodiments, active compounds may be administered orally. This is contemplated for agents which are generally resistant, or have been rendered resistant, to proteolysis by digestive enzymes. Such compounds are contemplated to include all those compounds, or drugs, that are available in tablet form from the manufacturer and derivatives and analogues thereof.

For oral administration, the active compounds may be administered, for example, with an inert diluent or with an assimilable edible carrier, or they may be enclosed in hard or soft shell gelatin capsule, or compressed into tablets, or incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of the unit. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

Upon formulation, the compounds will be administered in a manner compatible with the dosage formulation and in such amount as is therapeutically effective. The formulations are easily administered in a variety of dosage forms, such as those described below in specific examples.

C. Liposomes

In a particular embodiment, liposomal formulations are contemplated. Liposomal encapsulation of pharmaceutical agents prolongs their half-lives when compared to conventional drug delivery systems. Because larger quantities can be protectively packaged, this allow the opportunity for dose-intensity of agents so delivered to cells. This would be particularly attractive in the chemotherapy of cervical cancer if there were mechanisms to specifically enhance the cellular targeting of such liposomes to these cells.

“Liposome” is a generic term encompassing a variety of single and multilamellar lipid vehicles formed by the generation of enclosed lipid bilayers. Phospholipids are used for preparing the liposomes according to the present invention and can carry a net positive charge, a net negative charge or are neutral. Dicetyl phosphate can be employed to confer a negative charge on the liposomes, and stearylamine can be used to confer a positive charge on the liposomes. Liposomes are characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are cationic lipid-nucleic acid complexes, such as lipofectamine-nucleic acid complexes.

D. Anthracycline Dosages and Routes of Administration

Anthracyclines are broad-spectrum anti-tumor antibiotics produced by the Streptomyces species. Their chemical structure comprises a four-ring chromophore attached to the amino sugar, daunosamine. The chromophore is composed of three planar rings, which allow the drug to intercalate with DNA, thereby causing cytotoxicity. Important examples of anthracyclines include, daunorubicin also commercially known as doxorubicin and adriamycin; actinomycin D, idarubicin, epirubicin, amsacrine, mitoxiantrone.

Anthracyclines are typically administered parenterally, although some anthracyclines such as idarubicin, may be administered orally. The most common route of administration is intravenous. Pharmacokinetic studies have shown that after about 3 hours of administration, tissue levels exceed that of plasma, reaching tissue-to-plasma ratios as high as 100. Intracellular concentrations of the drug shown that greater than 80% is found within the nucleus. Thus, shortly after administration, bulk of the drug in the body is bound to DNA.

Majority of anthracycline metabolism is by the liver. Side chains are reduced to the corresponding alcohol, for example, daunorubicinol or doxorubicinol, within the liver. The plasma disappearance curve for anthracyclines is typically biphasic, with a rapid early distributive phase followed by a terminal phase with half-lives on the order of 24 to 48 hours due to slow release of drug bound to DNA. In the case of epirubicin, hepatic glucuronidation plays an important role in drug metabolism.

Anthracyclines dosages include, bolus administration every 28 days, once a week, daily for 3 to 4 days and by continuous infusion for various times as decided by the physician. Drug tolerance is relatively independent of schedule of administration, for example, 60 mg/m² of doxorubicin results in similar overall toxicity whether given by bolus or by 96-hour infusion. However, dose-limiting toxicities are seen, for example, bolus administration of doxorubicin, dose-limiting toxicity results in myelosuppression, while with a 96-hour infusion, mucositis becomes more of a problem. Clinical trials have indicated that prolonged infusions may be less cardiotoxic than large, monthly, bolus-dose administration.

Side-effects and Toxicity

The major side-effects or toxicities of the anthracyclines include myelosuppression, mucositis, hair loss, cardiac toxicity, and severe local injury on extravasation. Cardiac toxicity can manifest in two distinct clinical syndromes, the drugs can precipitate an acute myocarditis-pericarditis syndrome in which the patient develops rapidly progressive heart failure and arrhythmias that are associated with fever and pericarditis. The second type of cardiac toxicity is a gradual loss of myocardial function with cumulative dosage of anthracycline. Each anthracycline is different with respect to the dosage and degree of myocardial damage it can cause.

Myelosuppression is another common dose-limiting toxicity of anthracyclines. Typically, granulocytopenia occurs, although, lymphopenia, thrombocytopenia, and anemia also occur. Mucositis is yet another side effect which results in inflammation and ulceration of oropharynx, esophagitis, colitis, and occasionally, vulvitis. Another common side effect is extravasation injury which is a result of leakage of the anthracyclines into subcutaneous tissues resulting in local tissue necrosis. In severe cases, the resulting ulcer can continue to extend over many months, resulting in severe disability and even loss of a limb. Other than these hair loss is another common side effect.

Interactions with anthracyclines also sensitize normal tissues to radiation damage for example, doxorubicin increases the severity of radiation pneumonitis, increases exposure of the heart to greater than 2,000 cGy which effectively increases the cardiac toxicity. However, most anthracyclines may be readily co-administered with most other anticancer drugs without significant risks. Thus, anthracycline drugs can be used effectively as a part of combination chemotherapy regimens.

V. Kits

Various kits may be assembled as part of the present invention. A kit may contain components to assay for SNPs in UGT2B7 to evaluate the ability of a particular patient to glucuronidate epirubicin, and thus provide a clinician with a suggested dosage range for treatment of the patient with epirubicin. Such kits may contain reagents that allow for SNPs to be evaluated, such as primer sets to evaluate SNPs correlated with relevant phenotypic manifestations concerning glucuronidation of epirubicin. It is contemplated that any of the following primers (or pairs of primers) complementary or identical to any of all or part of SEQ ID NOS:3-78 may be part of a kit.

All the essential materials and reagents required for assaying for UGT2B7 SNPs by a particular method discussed above may be assembled together in a kit. When the components of the kit are provided in one or more liquid solutions, the liquid solution preferably is an aqueous solution, with a sterile aqueous solution being particularly preferred.

The components of the kit may also be provided in dried or lyophilized forms. When reagents or components are provided as a dried form, reconstitution generally is by the addition of a suitable solvent. It is envisioned that the solvent also may be provided in another container means. The kits of the invention may also include an instruction sheet outlining suggested epirubicin dosages when particular SNPs are identified in a patient.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with, an instrument for assisting with sample collection and evaluation. Such an instrument may be an inhalant, syringe, pipette, forceps, measured spoon, eye dropper or any such medically approved delivery vehicle.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Materials and Methods

The following materials and methods were implemented with respect to Examples 2-9.

Chemicals and Reagents

Epirubicin was kindly provided by Pharmacia & Upjohn (Milan, Italy). Bovine serum albumin, daunorubicin, β-glucuronidase, magnesium chloride, tris(hydroxymethyl)amino-methane (Tris), and UDP-glucuronic acid (UDPGA) were purchased from Sigma (St. Louis, Mo.). Acetonitrile, hydrochloric acid, methanol, ortho-phosphoric acid, and sodium dihydrogen phosphate were obtained from Fisher Scientific Co. (Fairlawn, N.J.).

Microsomes Expressing Specific Human UGTs

Microsomes from human lymphoblasts and insect cells (BTI-TN-5B1-4) both transfected with a vector containing human UGT1A1, UGT1A3, UGT1A4, UGT1A6, UGT1A9 and UGT2B15 complementary DNA (cDNA) and their negative control (microsomes from cells infected with wild-type vector) were obtained from Gentest Corp. (Woburn, Mass.). Microsomes from insect cells (SF-9) infected with a baculovirus containing human cDNA for UGT2B7 and their negative control were purchased from PanVera (Madison, Wis.).

Preparation of Human Liver Microsomes

Normal human livers (n=47) were obtained through the Liver Tissue Procurement and Distribution System (National Institutes of Diabetes and Digestive and Kidney Diseases, Minneapolis, Minn.) after the approval of the Institutional Review Boards. Liver samples from Crigler-Najjar syndrome type I (CN-I) patients (n=2) were obtained from Children's Hospital and Queen Elizabeth Hospital (Birmingham, UK). Microsomes were prepared by differential centrifugation methods (Purba et al., 1987). Total protein content in microsomes was determined by the Bradford method using bovine serum albumin as the standard. Microsomes from normal human livers (n=47) were pooled for use in the optimization of glucuronidation reactions and kinetic analysis.

Epirubicin Glucuronidation Assay

A typical incubation consisted of final concentrations of epirubicin (200 μM), magnesium chloride (10 mM), total microsomal protein (3 mg/ml), and Tris-HCl buffer (0.1 M, pH 7.4) in a total volume of 100 μl. All mixtures were pre-incubated for 5 min at 37° C. to achieve thermal equilibrium and the reaction was initiated by adding UDPGA (5 mM). After 4 h of incubation in a shaking water bath at 37° C., the reaction was stopped with 0.4 ml of cold methanol. After the addition of 10 μl of the internal standard (daunorubicin, 1 nmole), samples were shaken for 20 min and centrifuged at 14,000 rpm for 30 min. The supernatant was dried under nitrogen at 37° C. and samples were resuspended with 200 μl of mobile phase. After centrifugation at 14,000 rpm for 15 min, the supernatant was injected into the high-pressure liquid chromatography (HPLC) system. Control reactions without epirubicin, microsomes, and UDPGA were simultaneously performed. Hydrolysis with β-glucuronidase was used to identify the epirubicin glucuronide peak. For this purpose, dried samples were reconstituted with 0.2 ml of sodium phosphate buffer (0.1 M, pH 6.8) containing 1000 U of β-glucuronidase (type VII, from E. coli) and incubated overnight at 37° C. Reference samples containing no enzyme were treated identically. The reaction was stopped with 0.4 ml of cold methanol and the two sets of samples were then analyzed as described below.

Owing to the lack of availability of pure epirubicin glucuronide, this metabolite was quantitated by comparison of measured peak heights to those of a standard curve generated for unchanged epirubicin. Fluorescence of epirubicin glucuronide was assumed to be equal to epirubicin based on their fluorescence spectra, similar to findings from other studies (Barker et al., 1996). The concentrations of epirubicin glucuronide were determined using a HPLC system (Hitachi Instruments, San Jose, Calif.) with fluorescence detection at 480 (λₑₓ) and 560 (λₑₘ) nm. Epirubicin, its glucuronide, and daunorubicin were separated using a reversed-phase Supelcosil LC-CN column (5 μm, 4.6×250 mm, Supelco Inc., Bellefonte, Pa.) preceded by a μBondapak LC-CN guardpak (Waters Corp., Milford, Mass.). The mobile phase consisted of 30% acetonitrile and 70% 50 mM sodium dihydrogen phosphate (pH adjusted to 4 with 8.5% ortho-phosphoric acid). At a flow of 0.8 ml/min, the retention times of epirubicin glucuronide, epirubicin, and daunorubicin were 5.7, 7.4, and 10.1 min, respectively. Standard curves for epirubicin were linear within the range of 5-800 μM. Inter-assay reproducibility was analyzed by incubating 3 pooled liver microsomal samples each day for 3 days, and the coefficient of variation was less than 5%. Intra-assay reproducibility was obtained by measuring epirubicin glucuronide formation in 10 separate incubations of the same batch of pooled liver microsomes, and the coefficient of variation was less than 5%.

Morphine Glucuronidation Assay

A typical incubation consisted of final concentrations of morphine (1.4 mM), magnesium chloride (5 mM), total microsomal protein (2 mg/ml), and Tris-HCl buffer (0.1 M, pH 7.4) in a total volume of 100 μl. After 5 min of pre-incubation at 37° C., the reaction was initiated by adding UDPGA (5 mM). After 20 min of incubation in a shaking water bath at 37° C., the reaction was stopped with 0.4 ml of cold acetonitrile. After the addition of 10 μl of the internal standard (10,11-dihydrocarbamazepine, 42 nmoles), samples were shaken for 20 min and centrifuged at 14,000 rpm for 30 min. The supernatant was dried under nitrogen at 37° C. and samples were resuspended with 200 μl of mobile phase. After centrifugation at 14,000 rpm for 15 min, the supernatant was injected into the HPLC system. Control reactions without morphine, microsomes, and UDPGA were simultaneously performed. The concentrations of morphine-3-glucuronide (M3G) and morphine-6-glucuronide (M6G) were determined by HPLC with fluorescence detection at 210 (λₑₓ) and 340 (λₑₘ) nm. Morphine, M3G, M6G, and 10,11-dihydrocarbamazepine were separated using a reversed-phase μBondapak C₁₈ column (10 μm, 3.9×300 mm, Waters Corp., Milford, Mass.) preceded by a Novapak C₁₈ guardpak (Waters Corp., Milford, Mass.). The mobile phase consisted of 25% acetonitrile and 75% 10 mM sodium dihydrogen phosphate and 1 mM sodium dodecyl sulfate (pH adjusted to 2.1 with 85% ortho-phosphoric acid). At a flow of 1 ml/min, the retention times of M3G, M6G, morphine, and 10,11-dihydrocarbamazepine were 8.9, 11.5, 17.1, and 27.7 min, respectively. Standard curves for M3G and M6G were linear within the range of 1-125 μM and 1-50 μM. Inter-assay reproducibility was analyzed by incubating 3 pooled liver microsomal samples each day for 3 days, and the coefficient of variation was 6.3% and 8.7% for M3G and M6G, respectively. Intra-assay reproducibility was obtained by measuring epirubicin glucuronide formation in 10 separate incubations of the same batch of pooled liver microsomes, and the coefficient of variation was 5.7% and 9.4% for M3G and M6G, respectively.

SN-38 Glucuronidation Assay

The measurement of glucuronidation rates of SN-38 in normal human liver microsomes (n=47) was performed as previously described (Iyer et al., 1998a).

Epirubicin Glucuronidation in HK293 Cell Membranes Expressing UGT2B7(H) and UGT2B7(Y) Variants

Two UGT2B7 variants have been identified, differing for a single amino acid change, i.e. tyrosine for histidine in UGT2B7(Y) and UGT2B7(H), respectively (Jin et al., 1993b). To test for possible differences in epirubicin glucuronidation rates between the two UGT2B7 variants, HK293 cells transfected with human cDNA and specifically expressing UGT2B7(Y) and UGT2B7(H) were used. Stable expression of human UGT2B7(Y) and UGT2B7(H) was obtained as previously described (Coffman et al., 1997). Membranes from HK293 cells were prepared according to the method described by King et al. (1997). Incubation conditions were those adopted for human liver microsomes.

Measurement of 7-ethoxycoumarin O-deethylation Activity

The measurement of 7-ethoxycoumarin O-deethylation (ECOD) activity in normal liver microsomes (n=47) was performed as previously published, using a substrate concentration of 1 mM (Evans and Relling, 1992).

Data Analysis and Statistics

Results are presented as mean±standard deviation (SD) of a single experiment performed in triplicate. In order to describe the formation rate of epirubicin glucuronide, pooled liver microsomes and UGT2B7 microsomes were separately incubated in the presence of a substrate range of 50-1000 μM, while the concentration of UDPGA was held constant (5 mM). Kinetics of conjugation reactions for morphine has been evaluated as well, and substrate concentration was varied from 0.2 to 10 mM. Two separate experiments in triplicate were performed. Data were analyzed by simple hyperbolic function (with r² indicating the goodness of fitting) and apparent Kₘ and Vₘₐₓ values of the reactions were estimated (GraphPad software, GraphPad Software Inc., San Diego, Calif.). Catalytic efficiencies (Vₘₐₓ/Kₘ) were also calculated. The Pearson correlation coefficient was adopted to test the level of correlation between epirubicin and other UGT substrates like morphine and SN-38, and the cut-off for statistical significance was set at 0.05. Frequency distribution of epirubicin glucuronidation in 47 microsomal preparations from normal human livers was described.

### Example 2

Optimization of Epirubicin and Morphine Glucuronidation Reaction

Optimal assay conditions were established using pooled liver microsomes. Variables such as incubation time, microsomal protein content, and UDPGA concentrations were examined. The enzymatic reaction was shown to be linear up to 30 min and 4 h of incubation for morphine and epirubicin, respectively. Maximal rates of morphine and epirubicin glucuronidation were obtained with a microsomal protein concentration of 2 mg/ml and 3 mg/ml, respectively. Increases in UDPGA concentration from 5 to 15 mM did not significantly change the production of glucuronidated metabolites of both drugs, and an UDPGA concentration of 5 mM was adopted.

### Example 3

Epirubicin Glucuronidation in Normal and CN-I Liver Microsomes

The formation rate of epirubicin glucuronide normal liver microsomes was 138±37 (mean±SD) pmol/min/mg (n=47) (Table 7). A coefficient of variation of 24% and a 4-fold difference were observed. In order to identify the possible contribution of UGT1A1 to epirubicin glucuronidation, the formation of epirubicin glucuronide was measured in CN-I liver microsomes. Glucuronidating activity of UGT1A1 is genetically absent in patients affected by CN-I, a severe unconjugated hyperbilirubinemia (Seppen et al., 1994). In liver microsomes from two CN-I patients, epirubicin glucuronidation rates were 104±6 pmol/min/mg and 144±6 pmol/min/mg (Table 7). These values are similar to the mean epirubicin glucuronidation observed in normal liver microsomes (Table 7).

### Example 4

Epirubicin Glucuronidation in Microsomes Expressing Human UGT cDNA

The screening of epirubicin glucuronidation activity in all commercially available microsomes expressing specific UGT isoforms revealed that epirubicin was glucuronidated only by UGT2B7. No epirubicin glucuronidating activity was observed in microsomes from cells expressing UGT1A1, UGT1A3, UGT1A4, UGT1A6, UGT1A9 and UGT2B15 (Table 7).

The formation rate of epirubicin glucuronide by cDNA expressed UGT2B7 was 63±4 pmol/min/mg (Table 7). There was no glucuronidation of epirubicin in control microsomes from cells infected with wild-type vector. The epirubicin glucuronide peak produced by cDNA expressed UGT2B7 was further confirmed by treatment with β-glucuronidase enzyme, which resulted in the loss of the glucuronide. Differences in epirubicin glucuronidation between UGT2B7(H) and UGT2B7(Y) variants were not observed, with mean±standard error values of 0.762±0.037 and 0.743±0.047 epirubicin glucuronide/internal standard, respectively.

TABLE 7
Formation rates of epirubicin glucuronide in liver microsomes from
normal individuals (n = 47), CN-I patients (n = 2), and microsomes
expressing specific UGT isoforms. Values are expressed as the mean ± SD
of a single experiment performed in triplicate. Epirubicin glucuronidation
in normal liver microsomes is the mean ± SD of 47 individuals.
Nd, not detectable.

| | Epirubicin glucuronide |
|---|---|
| Source | (pmol/min/mg) |
| Normal livers | 138 ± 37 |
| CN-I n.1 | 144 ± 6 |
| CN-L n.2 | 104 ± 6 |
| UGT2B7 | 63 ± 4 |
| UGT2B15 | Nd |
| UGT1A1 | Nd |
| UGT1A3 | Nd |
| UGT1A4 | Nd |
| UGT1A6 | Nd |
| UGT1A9 | Nd |

### Example 5

Kinetic Parameters and Frequency Distribution of Epirubicin Glucuronidation in Human Liver Microsomes

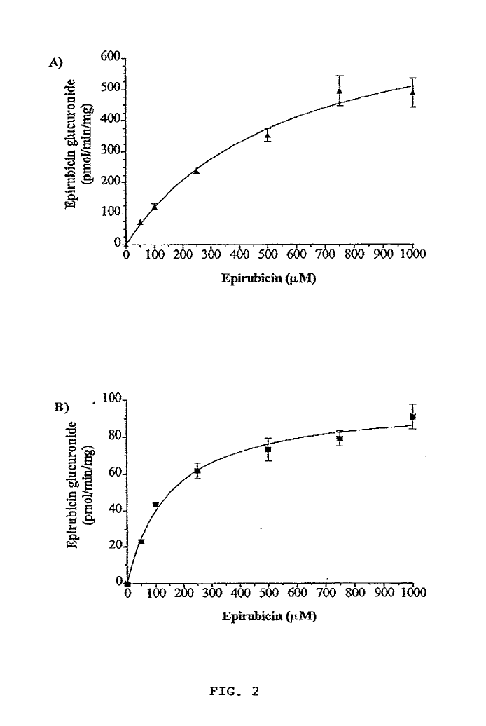

Formation rate of epirubicin glucuronide as a function of substrate concentration was measured in pooled human liver microsomes and in microsomes expressing UGT2B7 (FIGS. 2A and 2B). Both reactions followed Michaelis-Menten kinetics (r²=0.99). In human liver microsomes, apparent Kₘ and Vₘₐₓ values were 568±130 μM and 798±87 pmol/min/mg (mean±standard error), respectively. In microsomes expressing UGT2B7, apparent Kₘ and Vₘₐₓ values were 149±22 μM and 99±4 pmol/min/mg (mean±standard error), respectively. Catalytic efficiencies (Vₘₐₓ/Kₘ ratios) were 1.4 and 0.66 μl/min/mg for liver microsomes and microsomes expressing UGT2B7, respectively. This apparent difference can be explained by differences in lipid composition of microsomal membranes and amount of functional enzyme (Remmel and Burchell, 1993).

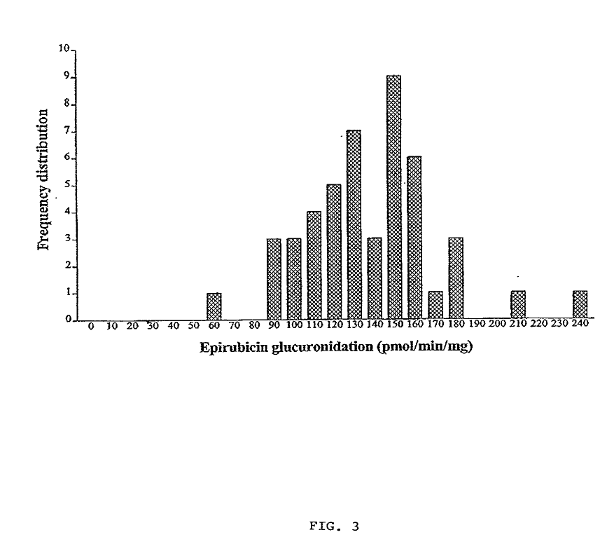

Frequency distribution analysis of epirubicin glucuronidation rates in 47 normal human liver microsomes showed that this phenotype is apparently normally distributed (FIG. 3). Median value of epirubicin glucuronidation rates was 136 pmol/min/mg, a value very close to the mean value (138 pmol/min/mg).

### Example 6

Kinetic Parameters of Morphine Glucuronidation in Human Liver Microsomes

The M3G and M6G glucuronidation rates were 1.25±0.46 and 0.19±0.06 (mean±SD) nmol/min/mg, with coefficients of variations of 37% and 32%, respectively. The M3G and M6G ratios were 6.55±0.89 (coefficient of variation of 13%), and the correlation coefficient between M3G and M6G was 0.92 (p<0.001). Both M3G and M6G formation followed Michaelis-Menten kinetics (r²=0.99 and 0.97 for M3G and M6G, respectively). With regard to M3G, apparent Kₘ and Vₘₐₓ values were 1988±225 μM and 1549±66 pmol/min/mg (mean±standard error), respectively. With regard to M6G, apparent Kₘ and Vₘₐₓ values were 1869±356 μM and 215±15 pmol/min/mg (mean±standard error), respectively. Catalytic efficiencies were 0.78 and 0.11 μl/min/mg for M3G and M6G, respectively (Table 8).

TABLE 8
Kinetic properties of epirubicin and morphine glucuronidation in human
liver microsomes. The kinetic properties of epirubicin glucuronidation in
baculosomes specifically expressing UGT2B7 are also indicated. Values
are expressed as the mean ± SE of two experiments performed in
triplicate.

| | Kₘ | Vₘₐₓ | Vₘₐₓ/Kₘ |
|---|---|---|---|
| | (μM) | (pmol/min/mg) | (μl/min/mg) |
| Epirubicin glucuronide | 149 ± 22 | 99 ± 4 | 0.66 |
| (insect baculosomes) | | | |
| Epirubicin glucuronide | 568 ± 130 | 798 ± 87 | 1.40 |
| (human liver microsomes) | | | |
| Morphine-3-glucuronide | 1988 ± 225 | 1549 ± 66 | 0.78 |
| (human liver microsomes) | | | |
| Morphine-6-glucuronide | 1869 ± 356 | 215 ± 15 | 0.11 |
| (human liver microsomes) | | | |

### Example 7

Correlation Study

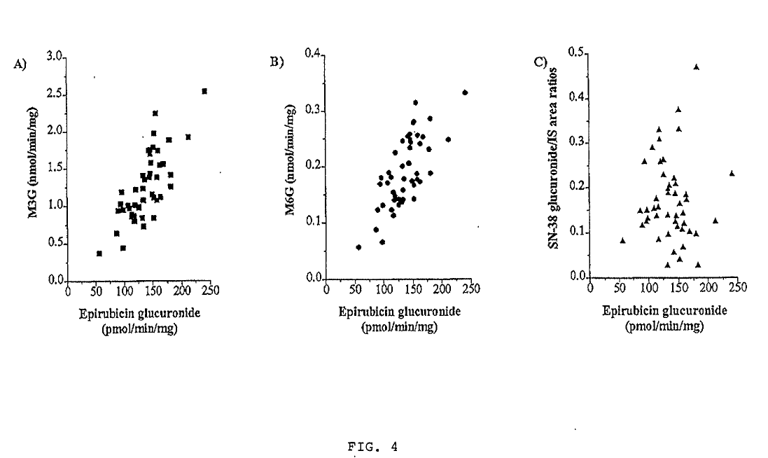

Since morphine is glucuronidated by UGT2B7 (Coffman et al., 1997), correlation between epirubicin and morphine glucuronidation rates was assessed in 47 normal human liver microsomes. Formation of epirubicin glucuronide was significantly related to that of M3G (r=0.76, p<0.001) and M6G (r=0.73, p<0.001) (FIGS. 4A and 4B, respectively). Correlation of glucuronidation rates between epirubicin and SN-38, the active metabolite of irinotecan and UGT1A1 substrate (Iyer et al., 1998b) was investigated. No correlation was observed with SN-38 glucuronidation (r=0.04) (FIG. 4C).

### Example 8

ECOD Activity

7-Ethoxycoumarin undergoes O-deethylation to umbelliferone by many different CYP450s, and the metabolism of 7-ethoxycoumarin can serve as an index of the proper handling and storage of the liver tissue and preparation of microsomes. ECOD activity in normal liver microsomes (n=47) ranged from 1.4 to 18.5 nmol/h/mg, similar to that previously reported (Relling et al., 1992).

### Example 9

Identification of UGT2B7 SNPs

The promoter region of the UGT2B7 gene was amplified using previously published sequence information (Ishii et al., and Genbank accession number NM_{—}001074). The primer sequences used for the promoter region amplification were 5′-GTGTCAATGGACTGCAGAAC-3′ (forward primer) and 5′-CCTTTCCACAATTCCCAGAG-3′ (reverse primer). The amplified product was sequenced in forward and reverse directions using the same primers as used for the amplification. Two SNPs were identified in 5 random DNA samples sequences. One was a T/C at position −161 and the other was T/C at -125.

### Example 10

Material and Methods

The following Materials and Methods were implemented with respect to Example 11.

Eligibility Criteria

Eligible patients were receiving patient-controlled (PCA) intravenous morphine sulfate under the supervision of the pain service of the University of Chicago Hospital; were at least 18 years old and able to provide informed consent. Patients over the age of 50 had a creatinine clearance greater than 50 mls/min. Patients with liver disease were eligible if their serum transaminases were less than 3 times the upper limit of normal (ULN) and if their bilirubin was less than 1.2 mg/dl. Patients were not enrolled if they had taken ranitidine in the prior week. Patients with a past history of orthotopic liver transplant were excluded.

Morphine Assay

Samples were drawn at approximately 24 and 26 hours after starting PCA Morphine. The heparinized blood samples were centrifuged and the plasma was stored at −70° C. until analysis.

Morphine-3-glucuronide (M3G), Morphine-6-Glucuronide (M6G), Morphine (M) and nalorphine were obtained from Sigma-Aldrich (St. Louis, Mo.). All other chemicals were of the highest grade available, and were purchased from Sigma-Aldrich (St. Louis, Mo.) and Fisher Scientific (Pittsburg, Pa.). Blank plasma was obtained from the Blood Bank at the University of Chicago Hospitals (Chicago, Ill.).

Plasma (1 ml) was combined with 170 μl of internal standard (5 μg/ml nalorphine in deionized water) and 4.5 ml of 0.5 M NaHCO₃. Solid phase extraction columns (Varian, BondElut C8, 3 ml, 500 mg) were conditioned with 10 ml of methanol, 5 ml of 40% acetonitrile in 10 mM sodium phosphate monobasic (pH 2.1), and 10 ml of deionized water. After loading the samples onto the columns, these were rinsed with 20 ml of 5 mM NaHCO₃, 0.5 ml of deionized water and 0.35 ml of 40% acetonitrile in 10 mM sodium phosphate monobasic (pH 2.1). The compounds of interest were eluted with 2 portions of 0.6 ml of 40% acetonitrile in 10 mM sodium phosphate monobasic (pH 2.1). After being evaporated to dryness using nitrogen gas (37° C.), the samples were reconstituted in 200 μl of mobile phase. Samples were centrifuged (15 min, 25° C., 14000 rpm) and 20 μl were injected onto the HPLC (Hitachi Instruments, San Jose, Calif.). The mobile phase consisted of 25/75 acetonitrile/10 mM sodium phosphate monobasic and 1 mM sodium dodecyl sulfate (pH 2.1) with a flow rate of 1 ml/min. A μBondapak C18 (10 μm, 3.9×300 mm ID) (Waters Corp, Milford, Mass.) and μBondapak guard-pak (Waters Corp, Milford, Mass.) were used. Fluorescence detection was used (λ excitation=210 nm, λ emission=340 nm). Retention times for M3G, M6G, M and nalorphine were 9, 12, 19 and 34 min, respectively (Bourquin et al., 1997).

UGT2B7 Promoter Sequencing and Genotyping for −161T/C Polymorphism

DNA was extracted from peripheral blood using a Puregene DNA isolation kit (Gentra system, Minneapolis, Minn.) according to the manufacturer's protocol. The promoter region was amplified by PCR using the following primers: forward—5′-GTGTCAATGGACTGCAGAAC-3′ (SEQ ID NO:3) and reverse —5′-CCTTTCCACAATTCCCAGAG-3′ (SEQ ID NO:4), which results in an amplified product of approximately 400 bp. The PCR reaction contained 1× PCR buffer with 2.5 mM MgCl₂ (Applied Biosystems), 0.2 mM each dNTP, 0.5 μM each primer and 1 U TaqGold polymerase (Applied Biosystems). PCR was performed at 95° C. for 10 mins followed by 35 cycles of 94° C. for 45 sec, 60° C. for 30 sec, 72° C. for 45 secs in a volume of 25 μl. PCR products were purified using the QIAquick PCR purification kit (Qiagen) and were cycle sequenced on both strands, using the same primers used for the PCR, using the BigDye Terminator chemistry (Applied Biosystems) following the manufacturer's recommended protocol. The sequence was analyzed using the Sequencher software from GeneCodes Corp.

For genotyping of the −161T/C polymorphism, a primer extension-based protocol using fluorescence polarization was performed (Chen et al., 1999), with some modifications as described in Hsu et al., 2001. PCR was performed using the same primers as described above for amplification of the promoter region. The PCR reaction contained 1× PCR buffer with 2.5 mM MgCl₂ (Qiagen), 0.5 mM each dNTP, 125 nM each primer and 0.25 U Hot Star Taq (Qiagen). PCR was performed at 95° C. for 15 mins followed by 40 cycles of 95° C. for 15 sec, 60° C. for 15 sec, 72° C. for 30 secs in a volume of 10 μl. PCR products were purified using shrimp alkaline phosphatase (Roche Biochemicals) and E. Coli exonuclease I enzymes (Amersham) followed by the primer extension reaction. The primer used for the single base extension was: 5′-TCTGAGCATGTGGATGGCAA-3′ (SEQ ID NO:71). The primer extension conditions used were those described by Hsu et al., 2001. Fluorescence polarization measurements were done on an LJL Analyst fluorescence reader (Molecular Devices Inc.).

UGT2B7 Exon 2-sequencing

Exon 2 was amplified by PCR using the following primers located in the flanking intron sequence: forward 5′-TGTCCGTATGCTACTATTGAA-3′ (SEQ ID NO:9) and reverse 5′-TGTGCTAATCCCTTTGTAAAT-3′ (SEQ ID NO:10) using the same PCR protocol as described for the promoter region. Sequence reactions were performed using the same forward primer as used for the PCR and the following reverse primer: 5′-GTTTGGCAGGTTTGCAGT GG-3′ (SEQ ID NO:72). Genotyping of the 802C/T (H268Y) polymorphism was performed by sequencing.

Data Analysis

Initially, UGT 2B7 was completely sequenced in the introns, exons and the 5′ and 3′ untranslated regions in the patients in the top and bottom deciles of the population distribution of M6G to Morphine ratio. The remaining population was then examined for new single nucleotide polymorphisms discovered in top and bottom deciles. The significance of a SNP was examined using the Jonckheere-Terpstra test using the whole population.

Linkage Disequilibrium refers to the tendency of specific combinations of alleles at two more linked loci to occur together on the same chromosorme more frequently than would be expected by chance. In 94 samples, the probability that the “C” allele at nucleotide −161 and the “C” allele at +802 occur together by chance, and vice versa for the “T” alleles is (0.5)⁹⁴. As this is highly improbable, it is therefore more likely that the two are linked. Complete LD refers to a 100% correlation between two alleles.

### Example 11

Polymorphism at −161 Correlates with Phenotype and is in Complete Linkage Disequilibrium with Polymorphism at Amino Acid 268

A total of 99 patients were enrolled from the University of Chicago Hospital pain service. The characteristics of the patients are listed in Table 9. No DNA was available for one sample, one sample was missing and no amplification was evident for three samples. Five samples had plasma interference and the levels of morphine and its metabolites could not be obtained. Thus phenotype and genotypes were available for 91 patients. One patient had undetectable morphine and could not be examined for the ratio of M6G to morphine, leaving 90 samples for the final analysis.

TABLE 9
Patient Characteristics

| Female/Male | 63/36 |
|---|---|
| Median Age (yrs) (range) | 51 (19-83) |
| Ethnic Origin | |
| Caucasian | 27 |
| African American | 68 |
| Hispanic | 2 |
| Asian | 2 |
| Median Creatinine mg/dl (range) | 0.8 (.5 to 1.5) |
| Median ALT U/L(range) | 14 (2 to 31) |
| Median Bilirubin mg/dl (range) | 0.4 (0.1 to 1) |

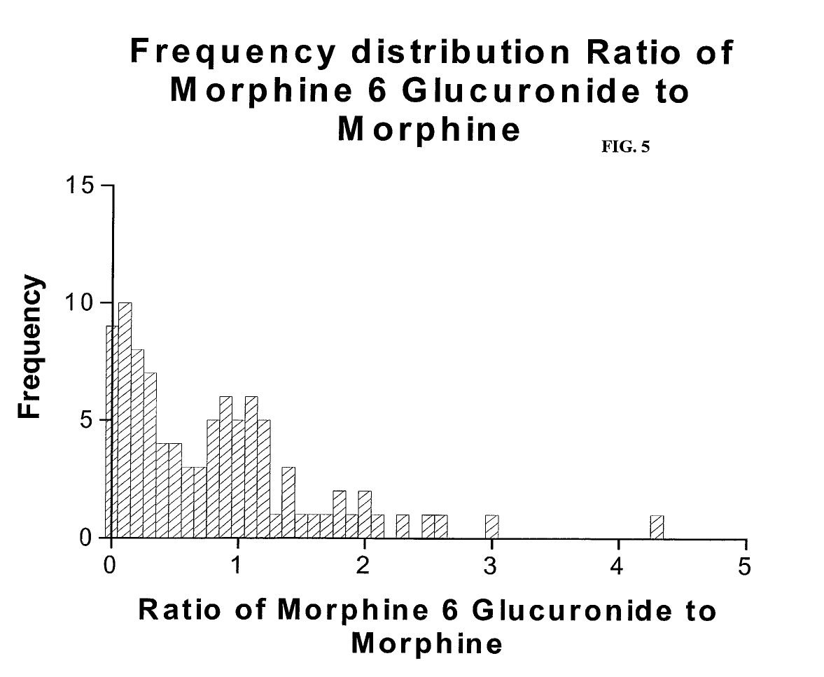

The concentration of morphine was 195±513 ng/ml (mean±standard deviation), M36 260±211 ng/ml, and M6G was 44±33 ng/ml. UGT2B7 is the uridine glucuronosyltransferase that glucuronidates at morphine at the 6 hydroxyl position; therefore we examined the ratio of morphine 6 glucuronide to morphine. The frequency distribution of the ratio of morphine-6-glucuronide to morphine is shown in FIG. 5. The UGT2B7 gene was sequenced in the top and bottom deciles of the preliminary population distribution. The introns, exons and the 5′ and 3′ untranslated region were sequenced. A new single nucleotide polymorphism, T to C at position −160, was discovered in the bottom decile of the population distribution of M6G to morphine (Table 10). The polymorphism at position −160 appeared to be in complete linkage disequilibrium (LD) with the known polymorphism at residue 268 in the coding region. The C SNP had a frequency of 55% and the T SNP had a frequency of 45%. The median ratios of M6G to M in the three genotypic groups were 0.311 (C/C), 0.755 (C/T) and 1.144 (T/T), which was statistically significant (Jonckheere-Terpstra test, p=0.004) (Table 11). The same test for a trend in the M3G/M ratio was significant (Jonckheere-Terpstra test, p=0.013).

TABLE 10
UGT2B7 in High and Low Outliers

| | Promoter Polymorphism-161 | |
|---|---|---|
| Sample Name | T/C | Ratio of M6G to M |
| T1 | T/C | 0.010471 |
| U | C/C | 0.014791 |
| B2 | T/C | 0.015136 |
| I | C/C | 0.016982 |
| V | C/C | 0.024547 |
| K2 | T/T | 2.041738 |
| E2 | C/C | 2.041738 |
| F2 | T/C | 2.344229 |
| H | T/T | 2.511886 |
| Z1 | T/T | 4.265795 |

TABLE 11
Ratios of M6G and M3G to Morphine

| | 25^{th} | | 75^{th} |
|---|---|---|---|
| | Percentile | Median | Percentile |
| M6G/M | | | |
| C/C | 0.59 | 0.311 | 1.039 |
| C/T | 0.224 | 0.755 | 1.265 |
| T/T | 0.467 | 1.144 | 1.943 |
| M3G/M | | | |
| C/C | 0.35 | 1.55 | 6.9 |
| C/T | 0.912 | 3.916 | 7.044 |
| T/T | 1.22 | 6.64 | 10.48 |

All of the COMPOSITIONS and METHODS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS and METHODS and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

References

The following references, to the extent that they provide exemplary procedural or other details supplementary to those set forth herein, are specifically incorporated herein by reference.

EPA No. 201,184

EPA No. 237,362

EPA No. 258,017

EPA No. 266,032

EPA No. 320 308

EPA No. 329 822

EPA No. 84,796

EPA No. 50,424

French Patent 2,650,840

PCT/US87/00880

PCT/US92/15712

U.S. Pat. No. 4,582,788

U.S. Pat. No. 4,656,127

U.S. Pat. No. 4,659,774

U.S. Pat. No. 4,682,195

U.S. Pat. No. 4,683,194

U.S. Pat. No. 4,683,195

U.S. Pat. No. 4,683,202

U.S. Pat. No. 4,684,611

U.S. Pat. No. 4,800,159

U.S. Pat. No. 4,816,571

U.S. Pat. No. 4,879,236

U.S. Pat. No. 4,883,750

U.S. Pat. No. 4,946,773

U.S. Pat. No. 4,952,500

U.S. Pat. No. 4,959,463

U.S. Pat. No. 5,141,813

U.S. Pat. No. 5,264,566

U.S. Pat. No. 5,279,721

U.S. Pat. No. 5,302,523

U.S. Pat. No. 5,322,783

U.S. Pat. No. 5,384,253

U.S. Pat. No. 5,428,148

U.S. Pat. No. 5,464,765

U.S. Pat. No. 5,538,877

U.S. Pat. No. 5,538,880

U.S. Pat. No. 5,550,318

U.S. Pat. No. 5,554,744

U.S. Pat. No. 5,563,055

U.S. Pat. No. 5,574,146

U.S. Pat. No. 5,578,832

U.S. Pat. No. 5,580,859

U.S. Pat. No. 5,589,466

U.S. Pat. No. 5,591,616

U.S. Pat. No. 5,602,244

U.S. Pat. No. 5,610,042

U.S. Pat. No. 5,645,897

U.S. Pat. No. 5,650,298

U.S. Pat. No. 5,656,610

U.S. Pat. No. 5,702,932

U.S. Pat. No. 5,705,629

U.S. Pat. No. 5,736,524

U.S. Pat. No. 5,780,448

U.S. Pat. No. 5,789,215

U.S. Pat. No. 5,840,873

U.S. Pat. No. 5,843,640

U.S. Pat. No. 5,843,650

U.S. Pat. No. 5,843,651

U.S. Pat. No. 5,843,663

U.S. Pat. No. 5,846,708

U.S. Pat. No. 5,846,709

U.S. Pat. No. 5,846,717

U.S. Pat. No. 5,846,726

U.S. Pat. No. 5,846,729

U.S. Pat. No. 5,846,783

U.S. Pat. No. 5,849,481

U.S. Pat. No. 5,849,483

U.S. Pat. No. 5,849,486

U.S. Pat. No. 5,849,487

U.S. Pat. No. 5,849,497

U.S. Pat. No. 5,849,546

U.S. Pat. No. 5,849,547

U.S. Pat. No. 5,851,770

U.S. Pat. No. 5,851,772

U.S. Pat. No. 5,853,990

U.S. Pat. No. 5,853,992

U.S. Pat. No. 5,853,993

U.S. Pat. No. 5,856,092

U.S. Pat. No. 5,858,652

U.S. Pat. No. 5,861,242

U.S. Pat. No. 5,861,244

U.S. Pat. No. 5,863,732

U.S. Pat. No. 5,863,753

U.S. Pat. No. 5,866,331

U.S. Pat. No. 5,866,337

U.S. Pat. No. 5,866,366

U.S. Pat. No. 5,871,986

U.S. Pat. No. 5,882,864

U.S. Pat. No. 5,900,481

U.S. Pat. No. 5,905,024

U.S. Pat. No. 5,910,407

U.S. Pat. No. 5,912,124

U.S. Pat. No. 5,912,145

U.S. Pat. No. 5,912,148

U.S. Pat. No. 5,916,776

U.S. Pat. No. 5,916,779

U.S. Pat. No. 5,919,626

U.S. Pat. No. 5,919,630

U.S. Pat. No. 5,922,574

U.S. Pat. No. 5,925,517

U.S. Pat. No. 5,925,525

U.S. Pat. No. 5,925,565

U.S. Pat. No. 5,928,862

U.S. Pat. No. 5,928,869

U.S. Pat. No. 5,928,870

U.S. Pat. No. 5,928,905

U.S. Pat. No. 5,928,906

U.S. Pat. No. 5,929,227

U.S. Pat. No. 5,932,413

U.S. Pat. No. 5,932,451

U.S. Pat. No. 5,935,791

U.S. Pat. No. 5,935,819

U.S. Pat. No. 5,935,825

U.S. Pat. No. 5,939,291

U.S. Pat. No. 5,942,391

U.S. Pat. No. 5,945,100

U.S. Pat. No. 5,952,174

U.S. Pat. No. 5,981,274

U.S. Pat. No. 5,994,624

U.S. Pat. No. 6,329,209

U.S. Pat. No. 6,329,140

U.S. Pat. No. 6,324,479

U.S. Pat. No. 6,322,971

U.S. Pat. No. 6,316,193

U.S. Pat. No. 6,309,823

U.S. Pat. No. 5,412,087

U.S. Pat. No. 5,445,934

U.S. Pat. No. 5,744,305

WO 84/03564

WO 88/10315

WO 89/06700

WO 90/07641

WO 91/02087

WO 94/09699

WO 95/06128

WO 00/06776

Almendro et al., J Immunol., 157:5411-5421, 1996.

Angel et al., Cell, 49:729, 1987b.

Angel et al., Mol. Cell. Biol., 7:2256, 1987a.

Atchison and Perry, Cell, 46:253, 1986.

Atchison and Perry, Cell, 48:121, 1987.

Ausubel, ed., Current protocols in molecular biology, New York, John Wiley & Sons, 1996.

Baichwal and Sugden, In: Gene Transfer, Kucherlapati R, ed., New York, Plenum Press, pp. 117-148, 1986.

Ballet et al., Pharmacol Res Commun 18:343-7, 1986.

Banerji et al., Cell, 27:299, 1981.

Banerji et al., Cell, 35:729, 1983.

Barker et al., J Chromatogr Biomed Appl 681:323-9, 1996.

Bellus, J. Macromol. Sci. Pure Appl. Chem, A31(1): 1355-1376, 1994.

Berkhout et al., Cell, 59:273, 1989.

Bhasker et al., Pharmacogenetics, 10(8):679-685, 2000.

Blanar et al., EMBO J., 8:1139, 1989.

Bodine and Ley, EMBO J., 6:2997, 1987.

Boshart et al., Cell, 41:521, 1985.

Bosze et al., EMBO J., 5:1615, 1986.

Bourquin et al., J. Chromatography B. 694:233-238, 1997.

Braddock et al., Cell, 58:269, 1989.

Bulla and Siddiqui, J. Virol., 62:1437, 1986.

Camaggi et al., Cancer Chemother Pharmacol 18:47-50, 1986.

Campbell and Villarreal, Mol. Cell. Biol., 8:1993, 1988.

Campere and Tilghman, Genes and Dev., 3:537, 1989.

Campo et al., Nature, 303:77, 1983.

Carbonelli et al. FEMS Microbiol Lett. 177(1):75-82, 1999.

Capaldi et al., Biochem. Biophys. Res. Comm., 76:425, 1977.

Celander and Haseltine, J. Virology, 61:269, 1987.

Celander et al., J. Virology, 62:1314, 1988.

Chandler et al., Cell, 33:489, 1983.

Chandler et al., Proc Natl Acad Sci USA. 94(8):3596-3601, 1997.

Chang et al., Mol. Cell. Biol., 9:2153, 1989.

Chatterjee et al., Proc. Nat'l Acad. Sci. USA., 86:9114, 1989.

Chen and Okayama, Mol. Cell Biol., 7:2745-2752, 1987.

Chen et al., Genome Res. 9:492-498, 1999.

Choi, et al., Cell, 53:519, 1988.

Cocea, Biotechniques. 23(5):814-816, 1997.

Coffman et al., Drug Metab Dispos 25:1-4, 1997.

Coffman et al., The Am. Soc. Pharmacology and Exp. Therapeutics, 26(1):73-77, 1998.

Coffman et al., Drug Metab Dispos 26:73-7, 1998.

Cohen et al., J. Cell. Physiol., 5:75, 1987.

Costa et al., Mol. Cell. Biol., 8:81, 1988.

Coupar et al., Gene, 68:1-10, 1988.

Cripe et al., EMBO J., 6:3745, 1987.

Culotta and Hamer, Mol. Cell. Biol., 9:1376, 1989.

Dandolo et al., J. Virology, 47:55, 1983.

DeRisi et al., Nature Genetics, 14:457-460, 1996.

De Villiers et al., Nature, 312:242, 1984.

Deschamps et al., Science, 230:1174, 1985.

Dobbs and Twelves, British Journal of Cancer, 78(f):662-666, 1998.

Edbrooke et al., Mol. Cell. Biol., 9:1908, 1989.

Edlund et al., Science, 230:912, 1985.

Evans and Relling, J Chromatogr 578:141-5, 1992.

Fechheimer et al., Proc. Nat'l Acad. Sci. USA, 84:8463-8467, 1987.

Feng and Holland, Nature, 334:6178, 1988.

Firak and Subramanian, Mol. Cell. Biol., 6:3667, 1986.

Foecking and Hofstetter, Gene, 45(1):101-5, 1986.

Fraley et al., Proc. Nat'l Acad. Sci. USA, 76:3348-3352, 1979.

Friedmann, Science, 244:1275-1281, 1989.

Froehler et al., Nucleic Acids Res. 14(13):5399-5407, 1986.

Frohman, In: PCR Protocols: A Guide To Methods And Applications, Academic Press, N.Y., 1990.

Fujita et al., Cell, 49:357, 1987.

Ghosh and Bachhawat, In: Liver diseases, targeted diagnosis and therapy using specific receptors and ligands, (Wu G, Wu C ed.), New York: Marcel Dekker, pp. 87-104, 1991.

Gilles et al., Cell, 33:717, 1983.

Gloss et al., EMBO J., 6:3735, 1987.

Godbout et al., Mol. Cell. Biol., 8:1169, 1988.

Goodbourn and Maniatis, Proc. Nat'l Acad. Sci. USA, 85:1447, 1988.

Goodbourn et al., Cell, 45:601, 1986.

Gopal, Mol. Cell Biol., 5:1188-1190, 1985.

Gossen and Bujard, Proc Natl Acad Sci USA, 89(12):5547-5551, 1992.

Gossen et al., Science, 268(5218):1766-1769, 1995.

Graham and Van Der Eb, Virology, 52:456-467, 1973.

Greene et al., Immunology Today, 10:272, 1989.

Grosschedl and Baltimore, Cell, 41:885, 1985.

Halushka et al., Nat. Genet., 22(3):239-247,1999.

Harland and Weintraub, J. Cell Biol., 101:1094-1099, 1985.

Haslinger and Karin, Proc. Nat'l Acad. Sci. USA., 82:8572, 1985.

Hauber and Cullen, J. Virology, 62:673, 1988.

Hen et al., Nature, 321:249, 1986.

Hensel et al., Lymphokine Res., 8:347, 1989.

Hermonat and Muzyczka, Proc. Nat'l. Acad. Sci. USA, 81:6466-6470, 1984.

Herr and Clarke, Cell, 45:461, 1986.

Hirochika et al., J. Virol., 61:2599, 1987.

Hirsch et al., Mol. Cell. Biol., 10:1959, 1990.

Holbrook et al., Virology, 157:211, 1987.

Horlick and Benfield, Mol. Cell. Biol., 9:2396, 1989.

Horwich et al. J. Virol., 64:642-650, 1990.

Hsu et al., BioTechniques, 31:560-570, 2001.

Huang et al., Cell, 27:245, 1981.

Hug et al., Mol Cell Biol., 8:3065, 1988.

Hwang et al., Mol. Cell. Biol., 10:585, 1990.

Imagawa et al., Cell, 51:251, 1987.

Imbra and Karin, Nature, 323:555, 1986.

Imler et al., Mol. Cell. Biol., 7:2558, 1987.

Imperiale and Nevins, Mol. Cell. Biol., 4:875, 1984.

Inazuka et al., Genome Res, 7(11):1094-1103, 1997.

Innis et al., Proc Natl Acad Sci USA. 85(24):9436-9440, 1988.

Ishii et al., Molecular Pharmacology 57:940-947, 2000.

Iyer et al., Ann Oncol 9:61 (abstract), 1998b.

Iyer et al., J Clin Invest 101:847-54, 1998a.

Jakobovits et al., Mol. Cell. Biol., 8:2555, 1988.

Jameel and Siddiqui, Mol. Cell. Biol., 6:710, 1986.

Jaynes et al., Mol. Cell. Biol., 8:62, 1988.

Jin et al., Biochem. Biphys. Res. Comm., 194(1):496-503, 1993.

Jin et al., J. Pharm. Experim. Therap., 264(1):475-479, 1993.

Johnson et al., Mol. Cell. Biol., 9:3393, 1989.

Kadesch and Berg, Mol. Cell. Biol., 6:2593, 1986.

Kaeppler et al., Plant Cell Reports 9: 415-418, 1990.

Kaneda et al., Science, 243:375-378, 1989.

Karin et al., Mol. Cell. Biol., 7:606, 1987.

Katinka et al., Cell, 20:393, 1980.

Katinka et al., Nature, 290:720, 1981.

Kato et al, J. Biol. Chem., 266:3361-3364, 1991.

Kawamoto et al., Mol. Cell. Biol., 8:267, 1988.

Kiledjian et al., Mol. Cell. Biol., 8:145, 1988.

King et al., Drug Metab Dispos 25:251-5, 1997.

Klamut et al., Mol. Cell. Biol., 10:193, 1990.

Koch et al., Mol. Cell. Biol., 9:303, 1989.

Komher, et al., Nucl. Acids. Res. 17:7779-7784, 1989.

Kraus et al. FEBS Lett., 428(3):165-170, 1998.

Kriegler and Botchan, In: Eukaryotic Viral Vectors, Y. Gluzman, ed., Cold Spring Harbor: Cold Spring Harbor Laboratory, NY, 1982.

Kriegler and Botchan, Mol. Cell. Biol., 3:325, 1983.

Kriegler et al., Cell, 38:483, 1984a.

Kriegler et al., Cell, 53:45, 1988.

Kriegler et al., In: Cancer Cells 2/Oncogenes and Viral Genes, Van de Woude et al. eds, Cold Spring Harbor, Cold Spring Harbor Laboratory, 1984b.

Kriegler et al., In: Gene Expression, D. Hamer and M. Rosenberg, eds., New York: Alan R. Liss, 1983.

Kuhl et al., Cell, 50:1057, 1987.

Kunz et al., Nucl. Acids Res., 17:1121, 1989.

Kuppuswamy, et al., Proc. Natl. Acad. Sci. (U.S.A.) 88:1143-1147,1991.

Kwoh et al., Proc. Nat. Acad. Sci. USA, 86: 1173, 1989.

Kwok et al., Genomics, 23(1):138-144, 1994.

Landegren, et al., Science 241:1077-1080, 1988.

Lareyre et al., J Biol Chem., 274(12):8282-8290, 1999.

Larsen et al., Proc. Nat'l Acad. Sci. USA., 83:8283, 1986.

Laspia et al., Cell, 59:283, 1989.

Latimer et al., Mol. Cell. Biol., 10:760, 1990.

Lee et al., J Auton Nerv Syst. 74(2-3):86-90, 1997.

Lee et al., Nature, 294:228, 1981.

Lee et al., Nucleic Acids Res., 12:4191-206, 1984.

Levenson et al., Hum Gene Ther. 20;9(8):1233-1236, 1998.

Levinson et al., Nature, 295:79, 1982.

Lin et al., Mol. Cell. Biol., 10:850, 1990.

Luria et al., EMBO J., 6:3307, 1987.

Lusky and Botchan, Proc. Nat'l Acad. Sci. USA., 83:3609, 1986.

Lusky et al., Mol. Cell. Biol., 3:1108, 1983.

Macejak and Sarnow, Nature, 353:90-94, 1991.

Mackenzie et al., Pharmacogenetics 7:255-69, 1997.

Mackenzie et al., Clin. Chem. Lab. Med., 38(9):869-892, 2000.

Majors and Varmus, Proc. Nat'l Acad. Sci. USA., 80:5866, 1983.

Maxam, et al., Proc. Natl. Acad. Sci. (U.S.A.) 74:560, 1977.

McNeall et al., Gene, 76:81, 1989.

Miksicek et al., Cell, 46:203, 1986.

Mordacq and Linzer, Genes and Dev., 3:760, 1989.

Moreau et al., Nucl. Acids Res., 9:6047, 1981.

Mross et al., J Clin Oncol 6:517-26, 1988.

Muesing et al., Cell, 48:691, 1987.

Mullis et al., Cold Spring Harbor Symp. Quant. Biol. 51:263-273, 1986.

Ng et al., Nuc. Acids Res., 17:601, 1989.

Nickerson et al., Proc. Natl. Acad. Sci. (U.S.A.) 87:8923-8927,1990.

Nicolas and Rubenstein, In: Vectors: A survey of molecular cloning vectors and their uses, Rodriguez and Denhardt (eds.), Stoneham: Butterworth, pp. 493-513, 1988.

Nicolau and Sene, Biochim. Biophys. Acta, 721:185-190, 1982.

Nicolau et al., Methods Enzymol., 149:157-176, 1987.

Nomoto et al., Gene, 236(2):259-271, 1999.

Nyren et al., Anal. Biochem. 208:171-175, 1993.

Ohara et al., Proc. Nat'l Acad. Sci. USA, 86: 5673-5677, 1989.

Omirulleh et al., Plant Mol. Biol., 21:415-28, 1993.

Ondek et al., EMBO J., 6:1017, 1987.

Ormrod et al., Drugs Aging 15:389-416, 1999.

Omitz et al., Mol. Cell. Biol., 7:3466, 1987.

Palmiter et al., Nature, 300:611, 1982.

Pech et al., Mol. Cell. Biol., 9:396, 1989.

Pelletier and Sonenberg, Nature, 334:320-325, 1988.

Perez-Stable and Constantini, Mol. Cell. Biol., 10:1116, 1990.

Picard and Schaffner, Nature, 307:83, 1984.

Pinkert et al., Genes and Dev., 1:268, 1987.

Ponta et al., Proc. Nat'l Acad. Sci. USA., 82:1020, 1985.

Porton et al., Mol. Cell. Biol., 10:1076, 1990.

Potrykus et al., Mol. Gen. Genet., 199:183-188, 1985.

Prezant et al., Hum. Mutat. 1:159-164, 1992.

Purba et al., Br J Clin Pharmacol 23:447-53, 1987.

Queen and Baltimore, Cell, 35:741, 1983.

Quinn et al., Mol. Cell. Biol., 9:4713, 1989.

Radominska-Pandya et al., Curr. Drug. Metab., 2:283-298, 2001.

Redondo et al., Science, 247:1225, 1990.

Reisman and Rotter, Mol. Cell. Biol., 9:3571, 1989.

Remington's Pharmaceutical Sciences, 15^{th} ed., pages 1035-1038 and 1570-1580, Mack Publishing Company, Easton, Pa., 1980.

Remmel and Burchell, Biochem Pharmacol 46:559-66, 1993.

Resendez Jr. et al., Mol. Cell. Biol., 8:4579, 1988.

Ridgeway, In: Vectors: A survey of molecular cloning vectors and their uses, Rodriguez R L, Denhardt D T, ed., Stoneham:Butterworth, pp. 467-492, 1988.

Ripe et al., Mol. Cell. Biol., 9:2224, 1989.

Rippe et al., Mol. Cell Biol., 10:689-695, 1990.

Rittling et al., Nucl. Acids Res., 17:1619, 1989.

Robert and Bui, Ann Oncol 3:651-6, 1992.

Robert, Clin. Pharmacokinet, 26:428, 1994

Rosen et al., Cell, 41:813, 1988.

Sakai et al., Genes and Dev., 2:1144, 1988.

Sambrook et al., In: Molecular Cloning: A Laboratory Manual, 2nd Ed., Cold Spring Harbor Press, Cold Spring Harbor, N.Y., 1989.

Sanger, et al., J. Molec. Biol., 94:441, 1975.

Satake et al., J. Virology, 62:970, 1988.

Schaffner et al., J. Mol. Biol., 201:81, 1988.

Schena, et al., Science, 270:467-470, 1995.

Schott and Robert Biochem. Pharmacol., 38:4069-74, 1989.

Searle et al., Mol. Cell. Biol., 5:1480, 1985.

Seppen et al., J Clin Invest 94:2385-91, 1994.

Sharp and Marciniak, Cell, 59:229, 1989.

Shaul and Ben-Levy, EMBO J., 6:1913, 1987.

Sherman et al., Mol. Cell. Biol., 9:50, 1989.

Sleigh and Lockett, J. EMBO, 4:3831, 1985.

Sokolov, Nucl. Acids Res. 18:3671, 1990.

Spalholz et al., Cell, 42:183, 1985.

Spandau and Lee, J. Virology, 62:427, 1988.

Spandidos and Wilkie, EMBO J., 2:1193, 1983.

Stephens and Hentschel, Biochem. J., 248:1, 1987.

Strassburg et al., J. Hepat. 34(6):865-872, 2001.

Stuart et al., Nature, 317:828, 1985.

Sullivan and Peterlin, Mol. Cell. Biol., 7:3315, 1987.

Swartzendruber and Lehman, J. Cell. Physiology, 85:179, 1975.

Syvanen et al., Genomics 8:684-692, 1990.

Takebe et al., Mol. Cell. Biol., 8:466, 1988.

Taillon-Miller et al., Genome Res, 8(7):748-754, 1998.

Taillon-Miller et al., Genome Res, 9(5):499-505, 1999.

Tavernier et al., Nature, 301:634, 1983.

Taylor and Kingston, Mol. Cell. Biol., 10:165, 1990a.

Taylor and Kingston, Mol. Cell. Biol., 10:176, 1990b.

Taylor et al., J. Biol. Chem., 264:15160, 1989.

Temin, In. Gene Transfer, Kucherlapati (ed.), New York: Plenum Press, pp. 149-188, 1986.

Thiesen et al., J. Virology, 62:614, 1988.

Tronche et al., Mol. Biol. Med., 7:173, 1990.

Tronche et al., Mol. Cell. Biol., 9:4759, 1989.

Trudel and Constantini, Genes and Dev., 6:954, 1987.

Tsumaki et al., J Biol Chem. 273(36):22861-22864, 1998.

Tyndall et al., Nuc. Acids. Res., 9:6231, 1981.

Ugozzoll et al., GATA 9:107-112, 1992.

Vannice and Levinson, J. Virology, 62:1305, 1988.

Vasseur et al., Proc. Nat'l Acad. Sci. USA., 77:1068, 1980.

Wade et al., Cancer Chemother. Pharmacol., 29:391, 1992.

Walker et al., Proc. Nat'l Acad. Sci. USA, 89:392-396 1992.

Wang and Calame, Cell, 47:241, 1986.

Weber et al., Cell, 36:983, 1984.

Weenen et al., Eur J Cancer Clin Oncol 20:919-26, 1984.

Weenen et al., Invest New Drugs 1:59-64, 1983.

Weinberger et al. Mol. Cell. Biol., 8:988, 1984.

Winoto and Baltimore, Cell, 59:649, 1989.

Wong et al., Gene, 10:87-94, 1980.

Wu et al., Biochem. Biophys. Res. Commun., 233(1):221-6, 1997.

Yutzey et al. Mol. Cell. Biol., 9:1397, 1989.

Zhao-Emonet et al., Biochem. Biophys. Acta., 1442(2-3):109-19, 1998.

What is claimed is:

1. A method for determining a dose of a UGT2B7-glucuronidated drug for a patient comprising:
a) determining the level of UGTB7 activity or expression in a patient;
b) determining the dose of the drug based on the level of UGT2B7 activity or expression in the patient.

2. The method of claim 1, wherein determining the level of UGT2B7 activity or expression comprises determining the nucleotide sequence at position −161 in one UGT2B7 gene of the patient.

3. The method of claim 1, wherein determining the level of UGT2B7 activity or expression comprises determining the nucleotide sequence at position +801 in one UGT2B7 gene of the patient.

4. The method of claim 1, wherein determining the level of UGT2B7 activity or expression comprises determining the nucleotide sequence at position +802 in one UGT2B7 gene of the patient.

5. The method of claim 2, further comprising:
c) classifying the UGT2B7 activity level in the patient, whereby identification of a thymidine residue indicates the patient does not have a low level of activity.

6. The method of claim 1, further comprising administering the drug to the patient.

7. The method of claim 2, wherein determining the nucleotide sequence at position −161 in the UGT2B7 gene comprises amplifying a sequence comprising position −161.

8. The method of claim 2, wherein determining the nucleotide sequence at position −161 in the UGT2B7 gene comprises sequencing a portion of the UGT2B7 promoter comprising position −161.

9. The method of claim 8, wherein position −161 is sequenced from one UGT2B7 promoter.

10. The method of claim 3, further comprising determining the nucleotide sequence at position −161 of a second UGT2B7 gene in the patient, whereby 1) identification of a second thymidine residue indicates a high level of UGT2B7 activity; 2) identification of a second cytosine residue indicates a low level of UGT2B7 activity; and, 3) identification of a residue different than the residue in the first promoter indicates an intermediate level of UGT2B7 activity.

11. The method of claim 2, wherein determining the nucleotide sequence at position −161 in one UGT2B7 gene comprises determining the nucleotide sequence of a first polymorphism in complete linkage disequilibrium (LD) with position −161 of the UGT2B7 gene.

12. The method of claim 11, wherein the nucleotide sequence of a polymorphism in complete LD is position +801 or +802 of the UGT2B7 gene.

13. The method of claim 12, wherein the nucleotide sequence at position +801 of the UGT2B7 gene is identified.

14. The method of claim 12, wherein the nucleotide sequence at position +802 of the UGT2B7 gene is identified.

15. The method of claim 12, wherein the nucleotide sequence at position +801 or +802 is a cytosine.

16. The method of claim 12, wherein the nucleotide sequence at position +801 or +802 is a thymidine.

17. The method of claim 11, wherein determining the nucleotide sequence of position −161 in one UGT2B7 gene further comprises determining the nucleotide sequence of a second polymorphism in complete linkage disequilibrium (LD) with the polymorphism at position −161 of the UGT2B7 gene.

18. The method of claim 17, wherein the second polymorphism in complete LD with the polymorphism at position −161 of the UGT2B7 gene is the polymorphism at position +801 or +802 of the UGT2B7 gene.

19. The method of claim 1, wherein the drug has an aliphatic carboxylic acid function.

20. The method of claim 19, wherein the drug is a propionic acid derivative, a phenylacetic acid derivative, a salicylic acid derivative, a acetic acid derivative, or an isobutyric acid derivative.

21. The method of claim 20, wherein the drug is a propionic acid derivative.

22. The method of claim 21, wherein the proprionic acid derivative is benoxaprofen, fenoprofen, ketoprofen, ibuprofen, naproxen, or tiaprofenic acid.

23. The method of claim 20, wherein the drug is a phenylacetic acid derivative.

24. The method of claim 23, wherein the phenylacetic acid derivative is etodolac, oxaprozin, or zomepirac.

25. The method of claim 20, wherein the drug is a salicylic acid derivative.

26. The method of claim 25, wherein the salicylic acid derivative is diflunisil.

27. The method of claim 20, wherein the drug is an acetic acid derivative.

28. The method of claim 27, wherein the acetic acid derivative is indomethacin, valproic acid, or zomepirac.

29. The method of claim 20, wherein the drug is an isobutyric acid derivative.

30. The method of claim 29, wherein the isobutyric acid derivative is clofibric acid.

31. The method of claim 1, wherein the drug is a polyhydroxylated estrogen.

32. The method of claim 31, wherein the polyhydroxylated estrogen is 4-hydroxyestrone, estriol, or 2-hydroxyestriol.

33. The method of claim 1, wherein the drug is a xenobiotic.

34. The method of claim 33, wherein the xenobiotic is 2-aminophenol, 4-OH biphenyl, androsterone, 1-naphthol, 4-methylumbelliferone, menthol, 4-nitrophenol, or hyodeoxycholic acid.

35. The method of claim 1, wherein the drug is an opioid.

36. The method of claim 35, wherein the opioid is morphinan derivative.

37. The method of claim 36, wherein the morphinan derivative is normorphine, norcodeine, codeine, naloxone, nalorphine, naltrexone, oxymorphone hydromorphone, dihydromorphone, levorphanol, nalmefene, naltrindole, naltriben, nalbuphine, or morphine.

38. The method of claim 35, wherein the opioid is an oripavine derivative.

39. The method of claim 38, wherein the oripavine derivative is norbuprenorphine, buprenorphine, or diprenorphine.

40. The method of claim 1, wherein the drug is propranolol, temazepam, chloramphenicol, oxazepam, androsterone, epitestosterone, zidovudine, or all-trans retinoic acid (ATRA).

41. The method of claim 1, wherein the drug is epirubicin or an epirubicin analog.

42. The method of claim 1, wherein the drug is a hydroxyl metabolite of an anthracycline.

43. A method of treating a patient with a UGT2B7-glucuronidated drug comprising:
a) determining the activity of UGT2B7 in a patient according to the method;
b) administering a dose of the drug to administer to the patient based on activity or expression level of UGT2B7.

44. The method of claim 43, wherein the drug has an aliphatic carboxylic acid function.

45. The method of claim 44, wherein the drug is a propionic acid derivative, a phenylacetic acid derivative, a salicylic acid derivative, a acetic acid derivative, or an isobutyric acid derivative.

46. The method of claim 45, wherein the drug is a propionic acid derivative.

47. The method of claim 46, wherein the proprionic acid derivative is benoxaprofen, fenoprofen, ketoprofen, ibuprofen, naproxen, or tiaprofenic acid.

48. The method of claim 45, wherein the drug is a phenylacetic acid derivative.

49. The method of claim 48, wherein the phenylacetic acid derivative is etodolac, oxaprozin, or zomepirac.

50. The method of claim 45, wherein the drug is a salicylic acid derivative.

51. The method of claim 50, wherein the salicylic acid derivative is diflunisil.

52. The method of claim 45, wherein the drug is an acetic acid derivative.

53. The method of claim 52, wherein the acetic acid derivative is indomethacin, valproic acid, or zomepirac.

54. The method of claim 45, wherein the drug is an isobutyric acid derivative.

55. The method of claim 54, wherein the isobutyric acid derivative is clofibric acid.

56. The method of claim 43, wherein the drug is a polyhydroxylated estrogen.

57. The method of claim 56, wherein the polyhydroxylated estrogen is 4-hydroxyestrone, estriol, or 2-hydroxyestriol.

58. The method of claim 43, wherein the drug is a xenobiotic.

59. The method of claim 58, wherein the xenobiotic is 2-aminophenol, 4-OH biphenyl, androsterone, 1-naphthol, 4-methylumbelliferone, menthol, 4-nitrophenol, or hyodeoxycholic acid.

60. The method of claim 43, wherein the drug is an opioid.

61. The method of claim 60, wherein the opioid is morphinan derivative.

62. The method of claim 61, wherein the morphinan derivative is normorphine, norcodeine, codeine, naloxone, nalorphine, naltrexone, oxymorphone hydromorphone, dihydromorphone, levorphanol, nalmefene, naltrindole, naltriben, nalbuphine, or morphine.

63. The method of claim 60, wherein the opioid is an oripavine derivative.

64. The method of claim 63, wherein the oripavine derivative is norbuprenorphine, buprenorphine, or diprenorphine.

65. The method of claim 43, wherein the drug is propranolol, temazepam, chloramphenicol, oxazepam, androsterone, epitestosterone, zidovudine, or all-trans retinoic acid (ATRA).

66. The method of claim 43, wherein the drug is epirubicin or an epirubicin analog.

67. The method of claim 43, wherein the drug is a hydroxyl metabolite of an anthracycline.

68. A method for evaluating the risk of toxicity of a UGT2B7-glucuronidated drug in a patient comprising:
a) identifying a patient at risk for toxicity from a UGT2B7-glucuronidated drug;
b) obtaining a sample from the patient;
c) determining the nucleotide sequence at position −161 in one UGT2B7 gene of the patient.

69. The method of claim 68, wherein the nucleotide sequence at position −161 in the other UGT2B7 gene of the patient is determined.

70. The method of claim 68, wherein the patient is a cancer patient.

71. The method of claim 70, wherein the drug is epirubicin or an epirubicin analog.

72. The method of claim 68, wherein the drug has an aliphatic carboxylic acid function.

73. The method of claim 72, wherein the drug is a propionic acid derivative, a phenylacetic acid derivative, a salicylic acid derivative, a acetic acid derivative, or an isobutyric acid derivative.

74. The method of claim 73, wherein the drug is a propionic acid derivative.

75. The method of claim 74, wherein the proprionic acid derivative is benoxaprofen, fenoprofen, ketoprofen, ibuprofen, naproxen, or tiaprofenic acid.

76. The method of claim 73, wherein the drug is a phenylacetic acid derivative.

77. The method of claim 76, wherein the phenylacetic acid derivative is etodolac, oxaprozin, or zomepirac.

78. The method of claim 73, wherein the drug is a salicylic acid derivative.

79. The method of claim 78, wherein the salicylic acid derivative is diflunisil.

80. The method of claim 73, wherein the drug is an acetic acid derivative.

81. The method of claim 80, wherein the acetic acid derivative is indomethacin, valproic acid, or zomepirac.

82. The method of claim 73, wherein the drug is an isobutyric acid derivative.

83. The method of claim 82, wherein the isobutyric acid derivative is clofibric acid.

84. The method of claim 68, wherein the drug is a polyhydroxylated estrogen.

85. The method of claim 84, wherein the polyhydroxylated estrogen is 4-hydroxyestrone, estriol, or 2-hydroxyestriol.

86. The method of claim 68, wherein the drug is a xenobiotic.

87. The method of claim 86, wherein the xenobiotic is 2-aminophenol, 4-OH biphenyl, androsterone, 1-naphthol, 4-methylumbelliferone, menthol, 4-nitrophenol, or hyodeoxycholic acid.

88. The method of claim 68, wherein the drug is an opioid.

89. The method of claim 88, wherein the opioid is morphinan derivative.

90. The method of claim 89, wherein the morphinan derivative is normorphine, norcodeine, morphine, codeine, naloxone, nalorphine, naltrexone, oxymorphone hydromorphone, dihydromorphone, levorphanol, nalmefene, naltrindole, naltriben, nalbuphine, or morphine.

91. The method of claim 88, wherein the opioid is an oripavine derivative.

92. The method of claim 91, wherein the oripavine derivative is norbuprenorphine, buprenorphine, or diprenorphine.

93. The method of claim 68, wherein the drug is propranolol, temazepam, chloramphenicol, oxazepam, androsterone, epitestosterone, zidovudine, or all-trans retinoic acid (ATRA).

94. A method for screening an individual for glucuronidation activity comprising
a) identifying a patient in need of screening for glucuronidation activity; and,
b) identifying the nucleotide sequence of a polymorphism that correlates with glucuronidation activity in the individual.

95. The method of claim 94, wherein the polymorphism is position −161, +801, or +802 in the UGT2B7 gene.

96. The method of claim 94, futher comprising obtaining a sample from the individual, wherein the sample comprises nucleic acid from the individual.

97. The method of claim 96, wherein the polymorphism is identified by amplifying the nucleic acid by PCR.

98. The method of claim 96, wherein the polymorphism is identified by sequencing the nucleic acid.

99. A method for prescribing a dose of a UGT2B7-glucuronidated drug to a patient comprising:
a) obtaining a sample from a patient in need of the UGT2B7-glucuronidated drug; and
b) determining the level of UGT2B7 glucuronidation in the patient.

100. A method for predicting the degree of an epirubicin-induced toxicity in a cancer patient comprising
a) identifying a cancer patient at risk for epirubicin-induced toxicity;
b) determining the nucleotide sequence at position −161 in both UGT2B7 alleles of the cancer patient.

101. A kit for evaluating the level of UGT2B7 activity in a subject comprising, in a suitable container means:
a) a first nucleic acid comprising 15 contiguous bases complementary or identical to the UGT2B7 gene, wherein the first nucleic acid allows the identification of the sequence of a first polymorphism in the UGT2B7 gene.

102. The kit of claim 101, wherein the first polymorphism is at position −161, +801, or +802 of the UGT2B7 gene.

103. The kit of claim 102, wherein the first polymorphism is at position −161.

104. The kit of claim 102, further comprising, in a suitable container means,
b) a second nucleic acid comprising 15 contiguous bases complementary or identical to the UGT2B7 gene, wherein the first nucleic acid allows the identification of the sequence of a second polymorphism in the UGT2B7 gene, in which the second polymorphism is a different than the first polymorphism.

105. The kit of claim 104, wherein the second polymorphism is at position −161, +801, or +802 of the UGT2B7 gene.

106. The kit of claim 105, further comprising, in a suitable container means,
b) a third nucleic acid comprising 15 contiguous bases complementary or identical to the UGT2B7 gene, wherein the first nucleic acid allows the identification of the sequence of a third polymorphism in the UGT2B7 gene, in which the third polymorphism is a different than the first and second polymorphisms.

107. The kit of claim 106, wherein the third polymorphism is at position −161, +801, or +802 of the UGT2B7 gene.

108. The kit of claim 107, wherein the first, second, and third nucleic acids are attached to a nonreactive array plate.


SEQUENCE LISTING (78 sequences)

SEQ ID NO: 1 - DNA, 1991 bp, Homo sapiens
  CDS (151)..(1740)
catgctcagactgttgatttaatgatattgtatgtactttgacttataagggttacattttaacttcttggctaatttatctttggacataaccatgagaaatgacagaaaggaacagcaactggaaaacaagcattgcattgcaccaggatgtctgtgaaatggacttcagtaattttgctaatacaactgagcttttgctttagctctgggaattgtggaaaggtgctggtgtgggcagcagaatacagccattggatgaatataaagacaatcctggatgagcttattcagagaggtcatgaggtgactgtactggcatcttcagcttccattctttttgatcccaacaactcatccgctcttaaaattgaaatttatcccacatctttaactaaaactgagttggagaatttcatcatgcaacagattaagagatggtcagaccttccaaaagatacattttggttatatttttcacaagtacaggaaatcatgtcaatatttggtgacataactagaaagttctgtaaagatgtagtttcaaataagaaatttatgaaaaaagtacaagagtcaagatttgacgtcatttttgcagatgctatttttccctgtagtgagctgctggctgagctatttaacataccctttgtgtacagtctcagcttctctcctggctacacttttgaaaagcatagtggaggatttattttccctccttcctacgtacctgttgttatgtcagaattaactgatcaaatgactttcatggagagggtaaaaaatatgatctatgtgctttactttgacttttggttcgaaatatttgacatgaagaagtgggatcagttttatagtgaagttctaggaagacccactacgttatctgagacaatggggaaagctgacgtatggcttattcgaaactcctggaattttcagtttcctcatccactcttaccaaatgttgattttgttggaggactccactgcaaacctgccaaacccctgcctaaggaaatggaagactttgtacagagctctggagaaaatggtgttgtggtgttttctctggggtcaatggtcagtaacatgacagaagaaagggccaacgtaattgcatcagccctggcccagatcccacaaaaggttctgtggagatttgatgggaataaaccagataccttaggtctcaatactcggctgtataagtggataccccagaatgaccttctaggtcatccaaagaccagagcttttataactcatggtggagccaatggcatctacgaggcaatctaccatgggatccctatggtggggattccattgtttgccgatcaacctgataacattgctcacatgaaggccaggggagcagctgttagagtggacttcaacacaatgtcgagtacagacttgctgaatgcattgaagagagtaattaatgatccttcatataaagagaatgttatgaaattatcaagaattcaacatgatcaaccagtgaagcccctggatcgagcagtcttctggattgaatttgtcatgcgccacaaaggagctaaacaccttcgggttgcagcccacgacctcacctggttccagtaccactctttggatgtgattgggttcctgctggtctgtgtggcaactgtgatatttatcgtcacaaaatgttgtctgttttgtttctggaagtttgctagaaaagcaaagaagggaaaaaatgattagttatatctgagatttgaagctggaaaacctgataggtgagactacttcagtttattccagcaagaaagattgtgatgcaagatttctttcttcctgagacaaaaaaaaaaaaaagaaaaaaaaatcttttcaaaatttactttgtcaaataaaaatttgtttttcagagatttaccacccagttcatggttagaaatattttgtggcaatgaagaaaacactacggaaaataaaaaataagataaagcctt

SEQ ID NO: 2 - protein, 529 aa, Homo sapiens
MSVKWTSVILLIQLSFCFSSGNCGKVLVWAAEYSHWMNIKTILDELIQRGHEVTVLASSASILFDPNNSSALKIEIYPTSLTKTELENFIMQQIKRWSDLPKDTFWLYFSQVQEIMSIFGDITRKFCKDVVSNKKFMKKVQESRFDVIFADAIFPCSELLAELFNIPFVYSLSFSPGYTFEKHSGGFIFPPSYVPVVMSELTDQMTFMERVKNMIYVLYFDFWFEIFDMKKWDQFYSEVLGRPTTLSETMGKADVWLIRNSWNFQFPHPLLPNVDFVGGLHCKPAKPLPKEMEDFVQSSGENGVVVFSLGSMVSNMTEERANVIASALAQIPQKVLWRFDGNKPDTLGLNTRLYKWIPQNDLLGHPKTRAFITHGGANGIYEAIYHGIPMVGIPLFADQPDNIAHMKARGAAVRVDFNTMSSTDLLNALKRVINDPSYKENVMKLSRIQHDQPVKPLDRAVFWIEFVMRHKGAKHLRVAAHDLTWFQYHSLDVIGFLLVCVATVIFIVTKCCLFCFWKFARKAKKGKND

SEQ ID NO: 3 - DNA, 20 bp, Homo sapiens
gtgtcaatggactgcagaac

SEQ ID NO: 4 - DNA, 20 bp, Homo sapiens
cctttccacaattcccagag

SEQ ID NO: 5 - DNA, 20 bp, Homo sapiens
cttggctaatttatctttgg

SEQ ID NO: 6 - DNA, 19 bp, Homo sapiens
cccactaccctgactttat

SEQ ID NO: 7 - DNA, 20 bp, Homo sapiens
ggacataaccatgagaaatg

SEQ ID NO: 8 - DNA, 19 bp, Homo sapiens
agctctgcttcaaagacac

SEQ ID NO: 9 - DNA, 21 bp, Homo sapiens
tgtccgtatgctactattgaa

SEQ ID NO: 10 - DNA, 21 bp, Homo sapiens
tgtgctaatccctttgtaaat

SEQ ID NO: 11 - DNA, 22 bp, Homo sapiens
tttttttttctattcctgtcag

SEQ ID NO: 12 - DNA, 16 bp, Homo sapiens
ctttaccccacccatt

SEQ ID NO: 13 - DNA, 20 bp, Homo sapiens
cccttgatctcattcctact

SEQ ID NO: 14 - DNA, 24 bp, Homo sapiens
aactggctattctttagatgtatg

SEQ ID NO: 15 - DNA, 25 bp, Homo sapiens
cattcctactctttatacagttctc

SEQ ID NO: 16 - DNA, 17 bp, Homo sapiens
cccccgattcagactat

SEQ ID NO: 17 - DNA, 20 bp, Homo sapiens
cccttgatctcattcctact

SEQ ID NO: 18 - DNA, 24 bp, Homo sapiens
aactggctattctttagatgtatg

SEQ ID NO: 19 - DNA, 18 bp, Homo sapiens
tcctccgaagtctgaaac

SEQ ID NO: 20 - DNA, 24 bp, Homo sapiens
tataaaaaaggatgaaactcacac

SEQ ID NO: 21 - DNA, 18 bp, Homo sapiens
caagcccccaagttatgt

SEQ ID NO: 22 - DNA, 20 bp, Homo sapiens
cagtaggatccgcgatataa

SEQ ID NO: 23 - DNA, 20 bp, Homo sapiens
tctgaggggttttgtctgta

SEQ ID NO: 24 - DNA, 20 bp, Homo sapiens
ccgcgatataagttcaacaa

SEQ ID NO: 25 - DNA, 20 bp, Homo sapiens
ggacataaccatgagaaatg

SEQ ID NO: 26 - DNA, 19 bp, Homo sapiens
ttaagagcggatgagttgt

SEQ ID NO: 27 - DNA, 20 bp, Homo sapiens
tcatcatgcaacagattaag

SEQ ID NO: 28 - DNA, 20 bp, Homo sapiens
cactacagggaaaaatagca

SEQ ID NO: 29 - DNA, 20 bp, Homo sapiens
accctttgtgtacagtctca

SEQ ID NO: 30 - DNA, 19 bp, Homo sapiens
agctctgcttcaaagacac

SEQ ID NO: 31 - DNA, 21 bp, Homo sapiens
ttgcctacattattctaaccc

SEQ ID NO: 32 - DNA, 17 bp, Homo sapiens
ctttaccccacccattt

SEQ ID NO: 33 - DNA, 25 bp, Homo sapiens
cattcctactctttatacagttctc

SEQ ID NO: 34 - DNA, 17 bp, Homo sapiens
cccccgattcagactat

SEQ ID NO: 35 - DNA, 25 bp, Homo sapiens
cattcctactctttatacagttctc

SEQ ID NO: 36 - DNA, 17 bp, Homo sapiens
cccccgattcagactat

SEQ ID NO: 37 - DNA, 18 bp, Homo sapiens
tcctccgaagtctgaaac

SEQ ID NO: 38 - DNA, 23 bp, Homo sapiens
tataaaaaggatgaaactcacac

SEQ ID NO: 39 - DNA, 20 bp, Homo sapiens
tctgaggggttttgtctgta

SEQ ID NO: 40 - DNA, 22 bp, Homo sapiens
ttttttgtctcaggaagaaaga

SEQ ID NO: 41 - DNA, 24 bp, Homo sapiens
aaaaaaagaaaaaaaaatcttttc

SEQ ID NO: 42 - DNA, 20 bp, Homo sapiens
ccgcgatataagttcaacaa

SEQ ID NO: 43 - DNA, 22 bp, Homo sapiens
tgcattgcaccaggatgtctgt

SEQ ID NO: 44 - DNA, 22 bp, Homo sapiens
tgcattgcaccaagatgtctgt

SEQ ID NO: 45 - DNA, 23 bp, Homo sapiens
tcctggatgagcttattcagaga

SEQ ID NO: 46 - DNA, 23 bp, Homo sapiens
tcctggatgagcctattcagaga

SEQ ID NO: 47 - DNA, 21 bp, Homo sapiens
cattttggttatatttttcac

SEQ ID NO: 48 - DNA, 21 bp, Homo sapiens
cattttggttttatttttcac

SEQ ID NO: 49 - DNA, 21 bp, Homo sapiens
cataactagaaagttctgtaa

SEQ ID NO: 50 - DNA, 21 bp, Homo sapiens
cataactaggaagttctgtaa

SEQ ID NO: 51 - DNA, 20 bp, Homo sapiens
cctggctacacttttgaaaa

SEQ ID NO: 52 - DNA, 20 bp, Homo sapiens
cctggctacatttttgaaaa

SEQ ID NO: 53 - DNA, 21 bp, Homo sapiens
gaagacccactacattatctg

SEQ ID NO: 54 - DNA, 21 bp, Homo sapiens
gaagacccactacgttatctg

SEQ ID NO: 55 - DNA, 26 bp, Homo sapiens
aattttcagtttccatatccactctt

SEQ ID NO: 56 - DNA, 26 bp, Homo sapiens
aattttcagtttcctcatccactctt

SEQ ID NO: 57 - DNA, 22 bp, Homo sapiens
taggtctcaatactcggctcta

SEQ ID NO: 58 - DNA, 22 bp, Homo sapiens
taggtctcaatactcggctgta

SEQ ID NO: 59 - DNA, 19 bp, Homo sapiens
tacaagtggataccccaga

SEQ ID NO: 60 - DNA, 19 bp, Homo sapiens
tataagtggataccccaga

SEQ ID NO: 61 - DNA, 26 bp, Homo sapiens
gggagaaagaatacattataattttt

SEQ ID NO: 62 - DNA, 25 bp, Homo sapiens
gggagaaagaatacttataattttt

SEQ ID NO: 63 - DNA, 21 bp, Homo sapiens
ttccattgtttgccgatcaac

SEQ ID NO: 64 - DNA, 21 bp, Homo sapiens
ttccattgtttgctgatcaac

SEQ ID NO: 65 - DNA, 21 bp, Homo sapiens
gaatgcattgaagagagtaat

SEQ ID NO: 66 - DNA, 21 bp, Homo sapiens
gaatgcattgcagagagtaat

SEQ ID NO: 67 - DNA, 22 bp, Homo sapiens
ctggtctgtgtggcaactgtga

SEQ ID NO: 68 - DNA, 22 bp, Homo sapiens
ctggtctgtgtggcgactgtga

SEQ ID NO: 69 - DNA, 19 bp, Homo sapiens
taagataaagccttatgag

SEQ ID NO: 70 - DNA, 19 bp, Homo sapiens
taagataaagacttatgag

SEQ ID NO: 71 - DNA, 20 bp, Artificial Sequence
  Description of Artificial Sequence  Synthetic Primer
tctgagcatgtggatggcaa

SEQ ID NO: 72 - DNA, 20 bp, Artificial Sequence
  Description of Artificial Sequence  Synthetic Primer
gtttggcaggtttgcagtgg

SEQ ID NO: 73 - DNA, 22 bp, Artificial Sequence
  Description of Artificial Sequence  Synthetic Primer
gaagcaaattctttcttcacag

SEQ ID NO: 74 - DNA, 21 bp, Artificial Sequence
  Description of Artificial Sequence  Synthetic Primer
accagtaaggcacttcatctt

SEQ ID NO: 75 - DNA, 22 bp, Artificial Sequence
  Description of Artificial Sequence  Synthetic Primer
cgattcagactataaagaatgt

SEQ ID NO: 76 - DNA, 17 bp, Artificial Sequence
  Description of Artificial Sequence  Synthetic Primer
tcctccgaagtctgaac

SEQ ID NO: 77 - DNA, 23 bp, Artificial Sequence
  Description of Artificial Sequence  Synthetic Primer
ccacctagtgaaaaatattgttc

SEQ ID NO: 78 - DNA, 21 bp, Artificial Sequence
  Description of Artificial Sequence  Synthetic Primer
atcacaatctttcttgctgga